SEQUENCE LISTING (45 sequences)

SEQ ID NO: 1 - DNA, 4103 bp, Hepatitis B virus
ttaatgcctttgtatgcatgtatacaagctaaacaggctttcactttctcgccaacttacaaggcctttctaagtaaacagcacatgaacctttaccccgttgctcggaaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcatcggaactgacaattctgtcgtcctctcgcggaaatatacatcgtttccatggctgctaggctgtactgccaactggatccttcgcgggacgtcctttgtttacgtcccgtcggcgctgaatcccgcggacgacccctcgcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgttagtattccttggactcataaggtgggaaactttacggggctttattcctctacagtacctatctttaatcctgaatggcaaactccttcctttcctaagattcatttacaagaggacattattaataggtgtcaacaatttgtgggccctctcactgtaaatgaaaagagaagattgaaattaattatgcctgctagattctatcctacccacactaaatatttgcccttagacaaaggaattaaaccttattatccagatcaggtagttaatcattacttccaaaccagacattatttacatactctttggaaggctggtattctatataagagggaaaccacacgtagcgcatcattttgcgggtcaccatattcttgggaacaagagctacagcatgggaggttggtcatcaaaacctcgcaaaggcatggggacgaatctttctgttcccaaccctctgggattctttcccgatcatcagttggaccctgcattcggagccaactcaaacaatccagattgggacttcaaccccatcaaggaccactggccagcagccaaccaggtaggagtgggagcattcgggccagggctcacccctccacacggcggtattttggggtggagccctcaggctcagggcatatggaccacagtgtcaacaattcctcctcctgcctccaccaatcggcagtcaggaaggcagcctactcccatctctccacctctaagagacagtcatcctcaggccatgcagtggaattccactgccttccaccaaactctgcaggatcccagagtcaggggtctgtatcttcctgctggtggctccagttcaggaacagtaaaccctgctccgaatattgcctctcacatctcgtcaatctccgcgaggactggggaccctgtgacgaacatggagaacatcacatcaggattcctaggacccctgctcgtgttacaggcggggtttttcttgttgacaagaatcctcacaataccgcagagtctagactcgtggtggacttctctcaattttctagggggatctcccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccagtacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaaaccctaacaaaacaaaaagatggggttattccctaaacttcatgggctacataattggaagttggggaactttgccacaggatcatattgtacaaaagatcaaacactgttttaaaaaacttcctgttaacaggcctattgattggaaagtatgtcaaaaaattgtgggtcttttgggctttgctgctccatttacacaatggggatatcctgccttaatgcctttgtatgcatgtatacaagctaaacaggctttcactttctcgccaacttacaaggcctttctaagtaaacagcacatgaacctttaccccgttgctcggaaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcatcggaactgacaattctgtcgtcctctcgcggaaatatacatcgtttccatggctgctaggctgtactgccaactggatccttcgcgggacgtcctttgtttacgtcccgtcggcgctgaatcccgcggacgacccctcgcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagc

SEQ ID NO: 2 - DNA, 4097 bp, Hepatitis B virus
ctaatgcctttatatgcatgtatacaagctaaacaggcttttactttctcgccaacttacaaggcctttctaaacaaacaatatctgaacctttaccccgttgctcggcaacggccaggtctgtgccaagtgtttgctgacgcaacccccactggctggggcttggccataggccatcagcgcatgcgtgggacctttgtgtctcctctgccgatccatactgcggaactcctagccgcttgttttgctcgcagcaggtctggagcgaaacttatcgggactgacaattccgttgtcctttcccgcaaatatacatcgtttccatggctgctaggctgtgctgccaactggatcctgcgcgggacgtcctttgtctacgtcccgtcggcgctgaatcccgcggacgacccctcccggggccgcttggggctctaccgcccgcttctccgtctgccgtaccgaccgaccacggggcgcacctctctttacgcggactccccgtctgtgccttctcgtctgccggaccgtgtgcacttcgcttcacctctgcacgtcgcatggagaccaccgtgaacgcccaccggaacttgcccaaggtcttgcataagaggactcttggactttcagcaatgtcaacgaccgaccttgaggcatacttcaaagactgtgtgtttaatgagtgggaggagttgggggaggagattaggttaaaggtctttgtactaggaggctgtaggcataaattggtctgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatcttctcgacaccgcctctgctctgtatcgggaggccttagagtctccggaacattgttcacctcaccatacggcactcaggcaagctattctgtgttggggtgagttaatgaatctagccacctgggtgggaagtaatttggaagacccagcatccagggaattagtagtcagctatgtcaatgttaatatgggcctaaaaatcagacaactattgtggtttcacatttcctgtcttacttttggaagagaaactgttcttgaatatttggtctcttttggagtgtggattcgcactcctcctgcatatagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtattccttggactcataaggtgggaaactttacggggctttattcttctacggtacctagctttaatcctaaatggcaaactccttcttttcctgacattcatttgcaggaggacattgttagtaggtgtaaacaatttgtgggacccctcacagtgaatgaaaacaggagactaaaattgattatgcctgctaggttctatcctaatgttactaaatatttgcccttagataaaggaattaaaccttattatccagagcatgtagttaatcattacttccagacgagacattatttacatactctttggaaggcgggtattttatataaaagagagacaacacgtagcgcctcattttgcgggtcaccatattcttgggaacaagagctacagcatgggaggttggtcctccaaacctcgacaaggcatggggacaaatctttctgtccccaatcctctgggattctttcccgatcaccagttggaccctgcattcaaagccaactccgacaatcccgattgggacctcaacccacacaaggacaactggccggactccaacaaggtgggagtgggagcattcgggccgggattcactccaccccatgggggactgttggggtggagccctcaagctcagggcatactcacaactgtgccagcagctcctcctcctgcctccaccaatcggcaggtaggaaggaagcctactcccctctctccacctctaagagacactcatcctcaggccatgcagtggaactccaccacgttccaccaaactcttcaagatccccgagtcagggctctgtactttcctgttggtggctccagttcagaaacagtgaaccctgttcagaacactgcctcttccatatcgtcaatcttatcgaagactggggaccctgtgccgaacatggagaacatcgcatcaggactcctaggacccctgctcgtgttacaggcggggtttttcttgttgacaaaaatcctcacaataccacagagtctagactcgtggtggacttctctcaattttctagggggaacacccgtgtgtcttggccaaaattcgcagtcccaaatctccagtcactcaccaacttgttgtcctccgatttgtcctggttatcgctggatgtgtctgcggcgttttatcatcttcctctgcatcctgctgctatgcctcatcttcttgttggttcttctggactatcaaggtatgttgcccgtttgtcctctaattccaggatcatcaaccaccagcaccggaccatgcaaaacctgcacgactcctgctcaaggaacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttatgccgctgttacccattttcttttgtctttgggtatacatctaaaccctcataaaacaaaacgatggggctactcccttaacttcatgggctatgtaattggaagttggggcaccttaccccaggaacatattgtgttgaaaatcaaacaatgttttaggaaacttcctgtaaacaggcctattgattggaaggtgtgtcaacgaattgtgggtcttttgggatttgctgctcctttcacacaatgtggttatcctgctctaatgcctttatatgcatgtatacaagctaaacaggcttttactttctcgccaacttacaaggcctttctaaacaaacaatatctgaacctttaccccgttgctcggcaacggccaggtctgtgccaagtgtttgctgacgcaacccccactggctggggcttggccataggccatcagcgcatgcgtgggacctttgtgtctcctctgccgatccatactgcggaactcctagccgcttgttttgctcgcagcaggtctggagcgaaacttatcgggactgacaattccgttgtcctttcccgcaaatatacatcgtttccatggctgctaggctgtgctgccaactggatcctgcgcgggacgtcctttgtctacgtcccgtcggcgctgaatcccgcggacgacccctcccggggccgcttggggctctaccgcccgcttctccgtctgccgtaccgaccgaccacggggcgcacctctctttacgcggactccccgtctgtgccttctcgtctgccggaccgtgtgcacttcgcttcacctctgcacgtcgcatggagaccaccgtgaacgcccaccggaacttgcccaaggtcttgcataagaggactcttggactttcagcaatgtcaacgaccgaccttgaggcatacttcaaagactgtgtgtttaatgagtgggaggagttgggggaggagattaggttaaaggtctttgtactaggaggctgtaggcataaattggtctgttcaccagcaccatgcaactttttcacctctgcctaatcatctcatgttcatgtcctactgttcaagcctccaagctgtgccttgggtggctttggggcatggacattgacccgtataaagaatttggagc

SEQ ID NO: 3 - DNA, 4097 bp, Hepatitis B virus
ttgatgcctctatatgcatgtatacaatctaagcaggctttcactttctcgccaacttacaaggcctttctgtgtaaacaatatctgaacctttaccccgttgcccggcaacggtcaggtctctgccaagtgtttgctgacgcaacccccactggatggggcttggccataggccatcagcgcatgcgtggaacctttgtggctcctctgccgatccatactgcggaactcctagcagcttgttttgctcgcagccggtctggagcaaaacttatcgggaccgacaactctgttgtcctctctcggaaatacacctcctttccatggctgctagggtgtgctgccaactggatcctgcgcgggacgtcctttgtctacgtcccgtcggcgctgaatcccgcggacgacccgtctcggggccgtttgggactctaccgtccccttcttcatctgccgttccggccaaccacggggcgcacctctctttacgcggtctccccgtctgtgccttctcatctgccggaccgtgtgcacttcgcttcacctctgcacgtcgcatggagaccaccgtgaacgcccaccaggtcttgcccaaggtcttacataagaggactcttggactctcagcaatgtcaacgaccgaccttgaggcatacttcaaagactgtgtgtttaaagactgggaggagttaggggaggagattaggttaaaggtctttgtactaggaggctgtaggcataaattggtctgttcatcagcaccatgcaactttttcacctctgcctaatcatctcatgttcatgtcctactgttcaagcctccaagctgtgccttgggtggctttagggcatggacattgacccttataaagaatttggagcatctgtggagttactctcttttttgccttctgacttctttccttctattcaagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagatccagcatccagggaattagtagtcagctatgtcaatgttaatatgggcctaaaaatcagacaactattgtggtttcacatttcctgtcttacttttggaagagaaactgttcttgagtatttggtatcttttggagtgtggattcgcactcctcccgcttacagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgagtggcaaactccctcctttcctcacattcatttacaggaagacattattaatagatgtcaacaatatgtgggccctcttacagttaatgaaaaaaggagattaaaattaattatgcctgctaggttctatcctaaccttactaaatatttgcccttggacaaaggcattaaaccgtattatcctgaacatgcagttaatcattacttcaaaactaggcattatttacatactctgtggaaggctggcattctatataagagagaaactacacgcagtgcctcattttgtgggtcaccatattcttgggaacaagagctacagcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcattggccagaggcaaatcaggtaggagcgggagcattcgggccagggttcaccccaccacacggcggtcttttggggtggagccctcaggctcagggcatattgacaacagtgccagcagcacctcctcctgcttccaccaatcggcagtcaggaagacagcctactcccatctctccacctctaagagacagtcatcctcaggccatgcagtggaactccacaacattccaccaagctctgctagatcccagagtgaggggcctatattttcctgctggtggctccagttccggaacagtaaaccctgttccgactactgcctcacccatatcgtcaatcttctcgaggactggggaccctgcaacgaacatggagaacacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaaccaccagcacggggccatgcaagacctgcacgattcctgctcaaggaacctctatgtttccctcttgttgctgtacaaaaccttcggatggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttaaaccctaataaaaccaaacgctggggctactcccttaacttcatgggatatgtaattggaagttggggtactttaccgcaggaacatattgtactaaaactcaagcaatgttttcgaaaattgcctgtaaatagacctattgattggaaagtatgtcaaagaattgtgggtcttttgggctttgctgccccttttacacaatgtggctatcctgcattgatgcctctatatgcatgtatacaatctaagcaggctttcactttctcgccaacttacaaggcctttctgtgtaaacaatatctgaacctttaccccgttgcccggcaacggtcaggtctctgccaagtgtttgctgacgcaacccccactggatggggcttggccataggccatcagcgcatgcgtggaacctttgtggctcctctgccgatccatactgcggaactcctagcagcttgttttgctcgcagccggtctggagcaaaacttatcgggaccgacaactctgttgtcctctctcggaaatacacctcctttccatggctgctagggtgtgctgccaactggatcctgcgcgggacgtcctttgtctacgtcccgtcggcgctgaatcccgcggacgacccgtctcggggccgtttgggactctaccgtccccttcttcatctgccgttccggccaaccacggggcgcacctctctttacgcggtctccccgtctgtgccttctcatctgccggaccgtgtgcacttcgcttcacctctgcacgtcgcatggagaccaccgtgaacgcccaccaggtcttgcccaaggtcttacataagaggactcttggactctcagcaatgtcaacgaccgaccttgaggcatacttcaaagactgtgtgtttaaagactgggaggagttaggggaggagattaggttaaaggtctttgtactaggaggctgtaggcataaattggtctgttcatcagcaccatgcaactttttcacctctgcctaatcatctcatgttcatgtcctactgttcaagcctccaagctgtgccttgggtggctttagggcatggacattgacccttataaagaatttggagc

SEQ ID NO: 4 - DNA, 4064 bp, Hepatitis B virus
ttaatgcccatttatgcaagtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaacctttctggctcctctgccgatccatactgcggaactcctagccgcttgttttgctcgcagccggtctggagcaaacattatcgggactgataactctgttgtcctctcccgcaaatatacatcgtttccatggctgctaggctgtgctgccaactggatcctgcgcgggacgtcctttgtttacgtcccgtcggcgctgaatcccgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatcttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacattgacccttataaagaatttggagctaccgtggagttactctcgtttttgccttctgacttctttccttcagtacgagatcttctagataccgcctcagctctgtatcgggaatccttagagtctcctgagcattgttcacctcaccatactgcactcaggcaagcaattctttgctggggggaactaatgactctagctacctgggtgggtgttaatttggaagatccagcatctagagacctagtagttagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattattaaaaaatgtgaacagtttgtaggcccactcacagttaatgagaaaagaagattgcaattgattatgcctgctaggttttatccaaaggttaccaaatatttacccttggataagggtattaaaccttattatccagaacatctagttaatcattacttccaaactagacactatttacacactctatggaaggcgggtatattatataagagagaaacaacacatagcgcttcattttgtgggtcacaatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcatccaccaatcgccagtcaggaaggcagcctaccccgctgtctccacctttgagaaacactcatcctcaggccatgcagtggaactccacaactttccaccaaactctgcaagatcccagagtgagaggcctgtatttccctgctggtggctccagttcaggaacagtaaaccctgttccgactactgcctctcccttatcgtcaatcttctcgaggattggggaccctgcgctgaacatggagagcatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcacgactcctgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatggggtatgtcattggatgttatgggtccttgccacaagaacacatcatacaaaaaatcaaagaatgttttagaaaacttcctattaacaggcctattgattggaaagtatgtcaacgaattgtgggtctcttaggttttgctgcccctttcacacaatgtggttatcctgctttaatgcccatttatgcaagtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaacctttctggctcctctgccgatccatactgcggaactcctagccgcttgttttgctcgcagccggtctggagcaaacattatcgggactgataactctgttgtcctctcccgcaaatatacatcgtttccatggctgctaggctgtgctgccaactggatcctgcgcgggacgtcctttgtttacgtcccgtcggcgctgaatcccgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatcttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacattgacccttataaagaatttggagc

SEQ ID NO: 5 - DNA, 261 bp, Hepatitis B virus
cacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccac

SEQ ID NO: 6 - DNA, 261 bp, Hepatitis B virus
cacgtcgcatggagaccaccgtgaacgcccaccggaacttgcccaaggtcttgcataagaggactcttggactttcagcaatgtcaacgaccgaccttgaggcatacttcaaagactgtgtgtttaatgagtgggaggagttgggggaggagattaggttaaaggtctttgtactaggaggctgtaggcataaattggtctgttcaccagcaccatgcaactttttcacctctgcctaatcatctcatgttcatgtcctac

SEQ ID NO: 7 - DNA, 261 bp, Hepatitis B virus
cacgtcgcatggagaccaccgtgaacgcccaccaggtcttgcccaaggtcttacataagaggactcttggactctcagcaatgtcaacgaccgaccttgaggcatacttcaaagactgtgtgtttaaagactgggaggagttaggggaggagattaggttaaaggtctttgtactaggaggctgtaggcataaattggtctgttcatcagcaccatgcaactttttcacctctgcctaatcatctcatgttcatgtcctac

SEQ ID NO: 8 - DNA, 261 bp, Hepatitis B virus
cacgtcgcatggagaccaccgtgaacgcccaccaaatcttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctac

SEQ ID NO: 9 - DNA, 261 bp, Hepatitis B virus
cacgtcgcatggaaaccaccgtgaacgcccaccaaatcttgcccaaggtcttatataagaggactcttggactctctgcaatgtcaacgaccgaccttgaggcatacttcaaagactgcttgtttaaagactgggaggagttgggggaggagattagattaatgatctttgtactaggaggctgtaggcataaattggtctgcgcaccagcaccatgcaactttttcacctctgcctaatcatctcttgttcatgtcctac

SEQ ID NO: 10 - DNA, 261 bp, Hepatitis B virus
cacgtcgcatggagaccaccgtgaacgccccttggagtttgccaacagtcttacataagaggactcttggactttcaggagggtcaatgacccggattgcagaatacatcaaagactgtgtatttaaggactgggaggagttgggggaggagactaggttaatgatctttgtactaggaggctgtaggcataaattggtctgttcaccagcaccatgcaactttttcacctctgcctaatcatcttttgttcatgtcctac

SEQ ID NO: 11 - DNA, 261 bp, Hepatitis B virus
cacgttacatggaaaccgccatgaacacctctcatcatctgccaaggcagttatataagaggactcttggactgtttgttatgtcaacaaccggggtggagaaatacttcaaggactgtgtttttgctgagtgggaagaattaggcaatgagtccaggttaatgacctttgtattaggaggctgtaggcataaattggtctgcgcaccagcaccatgtaactttttcacctctgcctaatcatctcttgttcatgtcctac

SEQ ID NO: 12 - DNA, 261 bp, Hepatitis B virus
cacgtcgcatggagaccaccgtgaacgccccttggaacttgccaacaaccttacataagaggactcttggactttcgccccggtcaacgacctggattgaggaatacatcaaagactgtgtatttaaggactgggaggagtcgggggaggagttgaggttaaaggtctttgtattaggaggctgtaggcataaattggtctgttcaccagcaccatgcaactttttcacctctgcctaatcatcttttgttcatgtcccac

SEQ ID NO: 13 - DNA, 1395 bp, Homo sapiens
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 14 - DNA, 1425 bp, Homo sapiens
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 15 - DNA, 1254 bp, Homo sapiens
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggaggtccgtgccaagcccaggaggggcggggttggagtggggactccccaggagacaggcctcacacagtgagctcacccctcagctccttggcttccccactgtgccgctttgggcaagttgcttaa

SEQ ID NO: 16 - DNA, 1404 bp, Homo sapiens
atgattttgttgccgctgcgtctcgccagattgaggcatcccctccgacatcactggagcatatctggaggggtggacagttctccacagggagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 17 - DNA, 1375 bp, Homo sapiens
atgattttgttgccgctgcgtctcgccagattgaggcatcccctccgacatcactggagcatatctggaggggtggacagttctccacagggagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggtctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 18 - DNA, 1233 bp, Homo sapiens
atgattttgttgccgctgcgtctcgccagattgaggcatcccctccgacatcactggagcatatctggaggggtggacagttctccacagggagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgcccaggaggggcggggttggagtggggactccccaggagacaggcctcacacagtgagctcacccctcagctccttggcttccccactgtgccgctttgggcaagttgcttaa

SEQ ID NO: 19 - DNA, 1329 bp, Homo sapiens
atggtcagcgtgaacgcgcccctcggggctccagtggagagttcttacgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 20 - DNA, 1359 bp, Homo sapiens
atggtcagcgtgaacgcgcccctcggggctccagtggagagttcttacgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 21 - DNA, 1188 bp, Homo sapiens
atggtcagcgtgaacgcgcccctcggggctccagtggagagttcttacgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggaggtccgtgccaagcccaggaggggcggggttggagtggggactccccaggagacaggcctcacacagtgagctcacccctcagctccttggcttccccactgtgccgctttgggcaagttgcttaa

SEQ ID NO: 22 - DNA, 1320 bp, Homo sapiens
atgtcggactggggccagggcttcccccaggacccaccagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 23 - DNA, 1350 bp, Homo sapiens
atgtcggactggggccagggcttcccccaggacccaccagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 24 - DNA, 1179 bp, Homo sapiens
atgtcggactggggccagggcttcccccaggacccaccagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggaggtccgtgccaagcccaggaggggcggggttggagtggggactccccaggagacaggcctcacacagtgagctcacccctcagctccttggcttccccactgtgccgctttgggcaagttgcttaa

SEQ ID NO: 25 - DNA, 20 bp, Artificial Sequence
  Probe
cgcagtatggatcggcagag

SEQ ID NO: 26 - DNA, 22 bp, Artificial Sequence
  Probe
gcacagcttggaggcttgaaca

SEQ ID NO: 27 - DNA, 22 bp, Artificial Sequence
  Probe
gagaagtccaccacgagtctag

SEQ ID NO: 28 - DNA, 22 bp, Artificial Sequence
  Probe
gatgaggcatagcagcaggatg

SEQ ID NO: 29 - DNA, 21 bp, Artificial Sequence
  Probe
cagaggtgaagcgaagtgcac

SEQ ID NO: 30 - DNA, 21 bp, Artificial Sequence
  Probe
tcccaccttatgagtccaagg

SEQ ID NO: 31 - DNA, 27 bp, Artificial Sequence
  Probe
ccttccaaagagtatgtaaataatgtc

SEQ ID NO: 32 - DNA, 25 bp, Artificial Sequence
  Primer
caccggcggtagtccacacagtgat

SEQ ID NO: 33 - DNA, 25 bp, Artificial Sequence
  Primer
aaacatcactgtgtggactaccgcc

SEQ ID NO: 34 - DNA, 25 bp, Artificial Sequence
  Primer
caccggtaactctcatagagatacg

SEQ ID NO: 35 - DNA, 25 bp, Artificial Sequence
  Primer
aaaccgtatctctatgagagttacc

SEQ ID NO: 36 - DNA, 20 bp, Artificial Sequence
  Guide RNA
gcggtagtccacacagtgat

SEQ ID NO: 37 - DNA, 20 bp, Artificial Sequence
  Guide RNA 2
gtaactctcatagagatacg

SEQ ID NO: 38 - DNA, 26 bp, Artificial Sequence
  Primer
ttctttcccgatcaccagttggaccc

SEQ ID NO: 39 - DNA, 21 bp, Artificial Sequence
  Primer
cccaccttgttggagtccggc

SEQ ID NO: 40 - DNA, 28 bp, Artificial Sequence
  Primer
ttaccagtgtgtatgccctcctaaatgg

SEQ ID NO: 41 - DNA, 26 bp, Artificial Sequence
  Primer
ccaggctcacatattccttgtcacag

SEQ ID NO: 42 - DNA, 25 bp, Artificial Sequence
  Primer
ctcctaaatgggtctatcttcttcc

SEQ ID NO: 43 - DNA, 28 bp, Artificial Sequence
  Primer
atattccttgtcacagtatttacagctg

SEQ ID NO: 44 - DNA, 26 bp, Artificial Sequence
  Primer
ttctttcccgatcatcagttggaccc

SEQ ID NO: 45 - DNA, 21 bp, Artificial Sequence
  Primer
cctacctggttggctgctggc

(19) United States
(12) Patent Application Publication
(10) Pub. No.: US 2022/0098682 A1
(43) Pub. Date: Mar. 31, 2022

(54) ENHANCED CELL BASED SCREENING PLATFORM FOR ANTI-HBV THERAPEUTICS

(71) Applicant: AGENCY FOR SCIENCE, TECHNOLOGY AND RESEARCH, Singapore (SG)

(72) Inventors: Ee Chee Ren, Singapore (SG); Hui Ling Ko, Singapore (SG)

(21) Appl. No.: 17/423,010

(22) PCT Filed: Jan. 14, 2020
(86) PCT No.: PCT/SG2020/050018

§ 371 Date: Jul. 14, 2021

(30) Foreign Application Priority Data
Jan. 14, 2019 (SG) 10201900356P

(51) Int. Cl. C12Q 1/70; C12N 15/63; C12N 7/00; C12N 5/071

(52) U.S. Cl. CPC C12Q 1/706; C12N 15/635; C12N 2710/00021; C12N 5/067; C12N 2500/30; C12N 7/00

(57) ABSTRACT

A cell comprising: a nucleotide sequence encoding a Hepatitis B Virus (HBV) operably linked to a promoter; two nucleotide sequences each encoding an isoform of HNF4α; and a nucleotide sequence encoding a repressor of HBV transcription, wherein said nucleotide sequence is mutated to decrease or silence expression of the repressor.

### CROSS-REFERENCE TO RELATED APPLICATIONS

This application claims the benefit of priority of Singapore application No. 10201900356P, filed 14 Jan. 2019, the contents of it being hereby incorporated by reference in its entirety for all purposes.

### FIELD OF THE INVENTION

The invention is in the field of cell biology. In particular, the present invention relates to a cell line for use in the screening of Hepatitis B virus (HBV) therapeutics.

### BACKGROUND OF THE INVENTION

Despite the availability of vaccines against the Hepatitis B virus (HBV) and anti-HBV therapeutics, Hepatitis B remains a major health problem with ˜300 million infected worldwide. Current antiviral therapies have resulted in poor clinical response as these therapeutic strategies are usually unable to achieve sustained off-treatment responses and eradicate the infection.

In vitro systems to model HBV infection are an imperative tool for studying HBV biology and for the discovery of new HBV therapeutics. New drugs for HBV therapy need to be identified and developed through large-scale screening of chemical libraries. However, the identification of effective drugs for HBV therapy is currently hampered by the lack of an efficient cell system that supports high efficiency HBV replication.

Primary human hepatocytes, which are considered to be the most physiologically relevant culture system for studying HBV biology in vitro, are expensive, scarce, vary among batches and have a limited life span and do not proliferate in culture. Induced human hepatocyte-like cells, while being virtually unlimited in supply, may not fully mimic mature hepatocytes. Human liver cell lines such as HepG2 and HuH7 have been widely used to study HBV replication as these are readily available and are robust in supporting steps in replication after transcription. However, the suboptimal efficiency of virus transcription in these cell lines has resulted in low levels of HBV viral replication, limiting the use of these cell systems in drug discovery, particularly in the screening of HBV therapeutics.

There is therefore a need to develop cell systems capable of high levels of HBV replication so as to provide a more sensitive testing system for the screening of HBV therapeutics.

### SUMMARY

In one aspect, there is provided a cell comprising: a nucleotide sequence encoding a Hepatitis B Virus (HBV) operably linked to a promoter; two nucleotide sequences each encoding an isoform of HNF4α; and a nucleotide sequence encoding a repressor of HBV transcription, wherein said nucleotide sequence is mutated to decrease or silence expression of the repressor.

In another aspect, there is provided a kit comprising the cell described herein together with instructions for use.

In another aspect, there is provided a method to produce HBV in vitro comprising culturing the cell described herein in the presence of an inducer for regulating transcription of the promoter.

In another aspect, there is provided a method of detecting the amount of HBV in a culture media in vitro comprising: culturing the cell described herein in a culture media comprising an inducer for regulating transcription of the promoter; contacting the cell with a probe capable of hybridizing to a target sequence on the HBV genome; hybridizing the probe to the target sequence, wherein a signal is emitted when the probe hybridizes to the target sequence; measuring the level of the emitted signal and comparing this to a signal from a reference sample to detect the amount of HBV in the culture media.

In another aspect, there is provided a method of identifying a HBV therapeutic agent comprising: culturing the cell described herein in a culture media comprising an inducer for regulating transcription of the promoter and the therapeutic agent; contacting the cell with a probe capable of hybridizing to a target sequence on the HBV genome; hybridizing the probe to the target sequence, wherein a signal is emitted when the probe hybridizes to the target sequence; measuring the level of the emitted signal and comparing this to a signal from a reference sample, wherein a decrease in the emitted signal compared to the reference sample identifies the HBV therapeutic agent.

### Definitions

As used herein, the term “Hepatitis B virus (HBV) genotype” refers to the genetic constitution of HBV. The 10 major HBV genotypes are genotypes A, B, C, D, E, F, G, H, I and J. Differences between HBV genotypes may explain variances in disease intensity, HBV replication efficiency and responses to antiviral treatment.

The term “Hepatitis B Virus core promoter (HBVCP)” refers to a region in the Hepatitis B viral genome that plays an important role for HBV replication. The HBVCP directs initiation of transcription for the synthesis of both the precore and pregenomic RNAs. The major functional elements of the HBVCP are the upper regulatory region and the basic core promoter. The HBVCP controls pregenomic RNA transcription, which is responsible for the synthesis of the core particle, which is necessary to produce infectious virions. The HBVCP also controls precore RNA transcription for Hepatitis B “e” antigen (HBeAg), which correlates with disease severity in carriers of HBV.

The term “HNF4α” in the context of a protein refers to a member of the nuclear receptor superfamily of ligand-dependent transcription factors. HNF4α may bind to DNA as homodimers or heterodimers. HNF4α is expressed in the liver, kidney, intestine and pancreas. The HNF4α protein is encoded by the HNFA gene. There are up to 12 different isoforms, HNF4α1 to HNF4α12, which differ at the N- and C-termini. Each HNF4α isoform heterodimer and isoform homodimer may regulate a distinct subset of genes in different tissues.

As used herein, the term “isoform” refers to a protein isoform which is a member of a set of structurally similar proteins that originate from a single gene or gene family. Protein isoforms may be formed as a result of alternative splicing, variable promoter usage, or post-transcriptional modifications of a single gene. The term “isoform” used in the context of HNF4α isoforms refers to protein isoforms of the HNFα proteins. HNFα isoforms result from both alternative splicing and alternate usage of promoters P1 and P2.

As used herein, the term “promoter” refers to a region of DNA that initiates transcription of a gene. A promoter may be a major promoter, a minor promoter or an alternative promoter. A major promoter is a promoter that is the most frequently used for the transcription of a gene. A promoter may be a constitutive promoter or an inducible promoter. A constitutive promoter is a promoter that is always active. An inducible promoter is a promoter that can be regulated in the presence of certain factors which may include certain biomolecules. An example of an inducible promoter system is the Tet-off system in which tetracycline and its derivatives serve as repressors of transcription. Another example of an inducible promoter system is the Tet-on system in which tetracycline and its derivatives serve as inducing agents to allow promoter activation.

As used herein, the term “operably linked” refers to the association of nucleic acid sequences on a single nucleic acid fragment so that the function of one is affected by the other. For example, a nucleotide sequence is said to be “operably linked” to a promoter if the two sequences are situated such that the promoter affects the expression of the nucleotide sequence (i.e., the nucleotide sequence is under the transcriptional control of the promoter). Coding sequences can be operably linked to regulatory sequences in sense or antisense orientation.

As used herein, the term “repressor” refers to a protein that has a negative effect on gene expression. The repressor binds to the operator region of a promoter and physically prevents the binding of proteins such as RNA polymerase, transcription factors, DNA-modifying proteins and chromatin-modifying proteins, thereby negatively influencing transcription of the gene. The repressor may also make transcription unfavourable by altering the 3D conformation of chromatin.

The term “stable integration” or “stably integrated” in the context of this application refers to the integration of foreign or exogenous DNA into the genome of a cell, preferably resulting in chromosomal integration and stable heritability through mitosis. A stable transformant is a cell which has stably integrated foreign DNA into the genomic DNA. A stable transformant is distinguished from a transient transformant in that, whereas foreign DNA is integrated into genomic DNA in the stable transformant, foreign DNA is not integrated into the genomic DNA in the transient transformant.

The term “CRISPR” in the context of CRISPR/Cas9 refers to Clustered Regularly Interspaced Short Palindromic Repeats. The CRISPR system is a gene editing technology which comprises a guide RNA and a CRISPR-associated Cas protein such as Cas9. In CRISPR/Cas9, the RNA-guided Cas9 nuclease from the CRISPR system can be used to facilitate genome engineering by specifying a targeting sequence within the guide RNA. The CRISPR system may be employed for a variety of genome editing methods including knocking out target genes, activating or repressing target genes, purifying specific regions of DNA and precisely editing DNA and RNA.

The term “hybridizing” as used herein refers to the ability of nucleic acids, such as probes or primers, of the present invention to bind to target nucleic acid sequences with sufficiently similar complementarity via complementary base strand pairing. Such hybridization may occur when nucleic acid molecules are contacted under appropriate conditions. A person skilled in the art would be familiar with parameters that affect hybridization; such as temperature, probe or primer length and composition, buffer composition and salt concentration and would be able to perform routine modification to adjust these parameters to achieve hybridization of a nucleic acid to a target sequence.

### BRIEF DESCRIPTION OF THE DRAWINGS

The invention will be better understood with reference to the detailed description when considered in conjunction with the non-limiting examples and the accompanying drawings, in which:

FIG. 1 shows the constructs used to generate Doxycycline-inducible HBV genotype B stable cell clones. In particular, (A) shows a Doxycycline-inducible construct for stably transfecting HBV genotype B replicon. (B) shows the functional domains of human Slug protein and the relative positions of mutations with successful SNAI2 gene disruption by selected guide RNAs and CRISPR/Cas9 targeting exon 2.

FIG. 2 shows that HBV genotype B has higher replication efficiency than other HBV genotypes in HuH7 cells. In particular, (A) shows that HBVCP from genotype B consistently generates significantly higher luminescence than other HBV genotypes, suggesting that HBV genotype B is the most efficient in HBV replication in HuH7 cells. (B) and (C) show that 1.3× full-length HBV replicons were compared for capacity to generate markers of HBV replication. In particular, (B) illustrates that HBV genotype B secretes most HBV envelope proteins (HBs) into the culture media, and this does not wane with time. (C) shows that HBV from genotype B also steadily secretes most Hepatitis B “e” antigen (HBeAg).

FIG. 3 shows the immunofluorescence staining of selected cell clones for HBs and HBc. The staining of HBc was significantly enhanced in Doxycycline-treated cells, providing confirmation that the inducible HBV replicon was completely integrated into the genome, allowing the Tet operator to enhance transcription at the HBVCP to generate more HBV nucleocapsid protein (HBc) in the presence of Doxycycline.

FIG. 4 shows that Slug knockout in HuH7 liver cell line is necessary for HNF4α-mediated enhancement in HBV production. In particular, (A) shows that HBV production is induced by the addition of 250 ng/mL doxycycline (Dox) every 48 hours, and results in significantly enhanced secretion of HBV rcDNA (relaxed circular DNA) into culture media. (B) shows that the addition of the potent HBV activator, HNF4α6 further enhances HBV production in the absence of Slug. In the presence of endogenous levels of Slug, clone C809 was insensitive to increase in HNF4α6 dose, restricting HBV production. Clone F881 overcame this limit in HBV synthesis as the absence of Slug permitted continually enhanced HBVCP transcription for sustained HBV replication hence enhanced rcDNA secretion with increasing HNF4α6 dose.

FIG. 5 shows that HNF4α isoform combinations significantly boost HBV production. In (A), cell clones were grown in 250 ng/mL doxycycline (Dox) in the presence of 100 ng/mL hygromycin and tetracycline-free culture media (DMEM) for 72 hours. Induced cells were then re-seeded at 1.6×10⁴ cells/well in 96-well plates, and transfected with 200 ng overexpression constructs for the indicated HNF4α isoform or isoform heterodimeric combinations in duplicates in the presence of 0.22 μL lipofectamine2000 per well. 24 hours later, the cells were gently washed and maintained for another 72 hours in tetratcycline-free culture media containing 250 ng/mL Doxycycline and 100 ng/mL hygromycin. In (B) and (C), culture media was harvested at 96 h post-transfection, and the amount of rcDNA copies/mL of culture media determined by quantitative real-time PCR. Amongst all possible HNF4α isoform homodimer and heterodimer combinations, HNF4α1-2 generated most HBV whereas HNF4α7-8 generated least HBV at 96 h post-transfection. Thus, the combination of Slug knockout in clone F881, Dox induction and overexpression of HNF4α1-2 results in a 32-fold increase in HBV production when compared to untreated HBV-producing cell clone C809 (C).

FIG. 6 shows a hybridization assay to rapidly detect HBV in culture media. Large amounts of HBV generated from the cell clones are readily detectable from a very small amount of culture media without the need for signal amplification and wash steps. Native molecular beacon probes keep their fluorescence reporter (5′ TYE™563) at the 5′ end quenched by close-proximity quenchers at the 3′ end (3′ IowaBlack® RQ) through their hairpin structure. When the probes are linearized by heat and bind specifically to target rcDNA sequences, the fluorophores are no longer in close proximity to the quenchers, hence emit fluorescence.

FIG. 7 shows a hybridization assay to rapidly detect HBV rcDNA in culture media. In particular, (A) shows the relative target positions of molecular beacon probes outlined in Table 1 with reference to the DNA cis elements of the HBV genome. Note that cccDNA is circular, hence not well re-presented in the schematic. HBV transcripts are also indicated. The (+) strand of rcDNA is incomplete and indicated by a dotted line. (B) shows that the molecular beacon probes bound specifically to rcDNA in culture media as fluorescence signal increased with probe concentration and saturates between 3-40 nM, whereas the culture media control containing no HBV did not have significant fluorescence readings and remained so without signal saturation at high probe concentrations exceeding 100 nM.

FIG. 8 shows that liver and non-liver cells were infected with HBV (genotype A), and HBV copies generated 72 hours post-infection was determined by quantitative real-time PCR of rcDNA found in infectious particles from 1 μl of culture media. Sequences of primers used: rcFA: 5′ ttctttcccgatcatcagttggaccc 3′ (SEQ ID NO: 44) and rcRA: 5′ CCTACCTGGTTGGCTGCTGGC 3′ (SEQ ID NO: 45). Several non-liver cells generated equivalent or more rcDNA than the liver cell lines, indicating that they produce equivalent or higher HBV titres than the liver cells.

FIG. 9 shows that liver and non-liver cells infected with HBV (genotype A) stain positive for HBV X protein (HBx) indicative of successful HBV entry and cccDNA formation to allow transcription of HBV products 3 days post-infection.

FIG. 10 shows that liver and non-liver cells infected with HBV (genotype A) stain positive for HBc indicative of continued active HBV replication and production 7 days post-infection.

### DETAILED DESCRIPTION OF THE PRESENT INVENTION

In a first aspect, the present invention refers to a cell comprising: a nucleotide sequence encoding a Hepatitis B Virus (HBV) operably linked to a promoter; two nucleotide sequences each encoding an isoform of HNF4α; and a nucleotide sequence encoding a repressor of HBV transcription, wherein said nucleotide sequence is mutated to decrease or silence expression of the repressor.

The HBV may be HBV of genotype A, B, C, D, E, F, G or H. Sequence variation between these genotypes may affect replication efficiency. The different HBV genotypes vary in transcription efficiency. The nucleotide sequence encoding HBV may be operably linked to the promoter in either a sense or antisense orientation.

It will generally be understood by a person skilled in the art that the transcription efficiency of different HBV genotypes varies with the cell type. In one embodiment, the HBV is HBV of genotype B.

In another embodiment, the promoter operably linked to the nucleotide sequence encoding the HBV is an inducible promoter. An inducible promoter may be regulated by positive or negative control. Inducible promoters include but are not limited to chemically inducible promoters, temperature inducible promoters, and light inducible promoters.

Inducible promoters include but are not limited to tetracycline-inducible promoters, cumate-inducible promoters, rapamycin-inducible promoters, abscisic acid-inducible promoters and light-inducible promoters. In one embodiment, the inducible promoter is a tetracycline inducible promoter. In one embodiment, the inducible promoter is a doxycycline inducible promoter.

In one embodiment, the nucleotide sequence encoding the HBV operably linked to a promoter is stably integrated into the genome of the cell.

In one embodiment, two nucleotide sequences each encoding an isoform of HNF4α are each operably linked to a promoter.

In another embodiment, the isoform of HNF4α is selected from the group consisting of HNF4α1, HNF4α2, HNF4α3, HNF4α4, HNF4α5, HNF4α6, HNF4α7, HNF4α8, HNF4α9, HNF4α10, HNF4α11 and HNF4α12.

In one embodiment, the present invention provides a cell as described herein wherein each of the two nucleotide sequences encodes the same isoform, or different isoforms of HNF4α. The isoforms may bind to DNA as homodimers or heterodimers.

In some embodiments, the nucleotide sequences described herein may encode isoforms HNF4α1 and HNF4α2 (HNF4α1-2), HNF4α2 and HNF4α3 (HNF4α2-3), HNF4α3 and HNF4α4 (HNF4α3-4), HNF4α2 and HNF4α6 (HNF4α2-6), HNF4α3 and HNF4α8 (HNF4α3-8), HNF4α4 and HNF4α8 (HNF4α4-8), HNF4α4 and HNF4α9 (HNF4α4-9), HNF4α6 and HNF4α12 (HNF4α6-12).

In one embodiment, the two nucleotide sequences encode isoforms HNF4α1 and HNF4α2 respectively.

In another embodiment, the mutation of the nucleotide sequence encoding a repressor of HBV transcription is selected from the group consisting of insertion, deletion, substitution or a combination thereof of one or more nucleotides.

In one embodiment, the repressor of HBV transcription is SLUG. SLUG is a member of the Snail family of zinc-finger transcription factors. It will generally be understood that SLUG is a transcriptional repressor that is encoded by the SNAI2 gene.

In one embodiment, the present invention provides a cell as described herein wherein the nucleotide sequence encoding SLUG is mutated or deleted. In another embodiment, the nucleotide sequence encoding SLUG is mutated at one or more positions in exon 2. In one embodiment, the nucleotide sequence encoding SLUG is mutated at one or more positions encoding amino acid residues starting from position 56 of SLUG. The mutation is selected from the group consisting of insertion, deletion, substitution or a combination thereof of one or more nucleotides. Methods for introducing mutations into the nucleotide sequence encoding SLUG are well known in the art.

In one embodiment, the nucleotide sequence encoding a repressor of HBV transcription is mutated by a CRISPR-Cas9 system. In one embodiment, the guide RNA of the CRISPR-Cas9 system is designed to target the SNAI2 gene. In another embodiment, the guide RNA is designed to target exon 2 of the SNAI2 gene.

In one embodiment, the cell as described herein is a hepatic cell. In another embodiment, the cell as described herein is a non-hepatic cell. Examples of non-hepatic cell include but are not limited to a colon cell, a pancreatic cell, a kidney cell, a breast cell, a stomach cell, a lung cell, a nerve cell, a muscle cell, a bone cell, a skin cell, an endothelial cell, a fat cell and a blood cell. In some embodiments, the non-hepatic cell is a colon cell, a pancreatic cell, a kidney cell or a breast cell.

In one embodiment, the cell is selected from the group consisting of HepG2, Huh7, Hep3B, Huh6, LS174T, RKO, HCT116, WiDr, Caco-2, HPAF II, A498, HEK293, MCF-7, AU565, A549 and Kato III cells. It will generally be understood that other suitable hepatic and non-hepatic cells may also be used in the present invention.

In one embodiment, the cell is HuH7.

In one embodiment, the cell is a cell line.

In one embodiment, the cell as described herein comprises HBV genotype B operably linked to a promoter, two nucleotide sequences each encoding an isoform of HNF4α, wherein the isoforms are HNF4α1 and HNF4α2; and a nucleotide sequence encoding a repressor of HBV transcription, wherein said nucleotide sequence is mutated to decrease or silence expression of the repressor, and wherein the repressor is SLUG.

In another embodiment, the cell as described herein is a hepatic cell comprising HBV genotype B operably linked to a promoter, two nucleotide sequences each encoding an isoform of HNF4α, wherein the isoforms are HNF4α3 and HNF4α4; and a nucleotide sequence encoding a repressor of HBV transcription, wherein said nucleotide sequence is mutated to decrease or silence expression of the repressor, and wherein the repressor is SLUG.

In another embodiment, the cell as described herein is a hepatic cell comprising HBV genotype B operably linked to a promoter, two nucleotide sequences each encoding an isoform of HNF4α, wherein the isoforms are HNF4α4 and HNF4α8; and a nucleotide sequence encoding a repressor of HBV transcription, wherein said nucleotide sequence is mutated to decrease or silence expression of the repressor, and wherein the repressor is SLUG.

In yet another embodiment, the cell as described herein is a hepatic cell comprising HBV genotype B operably linked to a promoter, two nucleotide sequences each encoding an isoform of HNF4α, wherein the isoforms are HNF4α4 and HNF4α9; and a nucleotide sequence encoding a repressor of HBV transcription, wherein said nucleotide sequence is mutated to decrease or silence expression of the repressor, and wherein the repressor is SLUG.

In yet another embodiment, the cell as described herein is a hepatic cell comprising HBV genotype B operably linked to a promoter, two nucleotide sequences each encoding an isoform of HNF4α, wherein the isoforms are HNF4α6 and HNF4α12; and a nucleotide sequence encoding a repressor of HBV transcription, wherein said nucleotide sequence is mutated to decrease or silence expression of the repressor, and wherein the repressor is SLUG.

In one aspect, the present invention refers to a kit comprising the hepatic cell as described herein together with instructions for use. The kit may further include one or more primers, probes, buffers and reagents.

In another aspect, the present invention provides a method to produce HBV in vitro comprising culturing the hepatic cell as described herein in the presence of doxycycline. The doxycycline may be present at the start of the method or subsequently added during the course of the method.

In one embodiment, the HBV is produced at an increased level compared to a baseline level. The baseline level is the level of HBV produced by a cell without the modifications described herein. In one embodiment, the baseline level is the level of HBV produced by a HuH7 cell without the modifications described herein.

In one aspect, the present invention refers to a method of detecting the amount of HBV in a culture media in vitro comprising: culturing the cell described herein in a culture media comprising an inducer for regulating transcription of the promoter; contacting the cell with a probe capable of hybridizing to a target sequence on the HBV genome; hybridizing the probe to the target sequence, wherein a signal is emitted when the probe hybridizes to the target sequence; measuring the level of the emitted signal and comparing this to a signal from a reference sample to detect the amount of HBV in the culture media.

In one embodiment, the inducer is doxycycline and the promoter is inducible by a Tet-on system.

The inducer may be present in the culture media at the start of the method or subsequently added during the course of the method.

In one embodiment, the probe comprises a nucleotide sequence that is complementary to the target sequence on the HBV genome. The probe may be a sense or antisense probe. In one embodiment, the probe is an antisense probe.

In one embodiment, the target sequence may include but is not limited to covalently closed circular DNA (cccDNA), HBV transcripts and relaxed circular DNA (rcDNA). In one embodiment, the target sequence is HBV rcDNA.

In one embodiment, the probe further comprises a detectable label at the 5′ end of the probe and a quencher on the 3′ end of the probe. In another embodiment, the detectable label is in close proximity with the quencher when the probe is not hybridized to the target sequence.

In one embodiment, the detectable label is a fluorophore.

In one embodiment, the probe is denatured by heat and subsequently hybridized to the target sequence at an optimal annealing temperature. In yet another embodiment, the signal emitted when the probe hybridizes to the target sequence is a fluorescence signal. In one embodiment, no fluorescence is emitted when the probe does not hybridize to the target sequence.

In yet another embodiment, the reference sample is a cell that does not produce HBV.

In one aspect, the present invention provides a method of identifying a HBV therapeutic agent comprising: culturing the cell described herein in a culture media comprising an inducer for regulating transcription of the promoter and the therapeutic agent; contacting the cell with a probe capable of hybridizing to a target sequence on the HBV genome; hybridizing the probe to the target sequence, wherein a signal is emitted when the probe hybridizes to the target sequence; measuring the level of the emitted signal and comparing this to a signal from a reference sample, wherein a decrease in the emitted signal compared to the reference sample identifies the HBV therapeutic agent.

The inducer and/or the therapeutic agent may be present in the culture media at the start of the method or subsequently added during the course of the method.

In one embodiment, the inducer is doxycycline and the promoter is inducible by a Tet-on system.

The therapeutic agent may be selected from the group consisting of a nucleic acid, nucleic acid analog, peptides, proteins, metal ions, hormones, small organic molecules and antimicrobial molecules, or a combination thereof.

In one embodiment, the probe comprises a nucleotide sequence that is complementary to the target sequence on the HBV genome. The probe may be a sense or antisense probe. In one embodiment, the probe is an antisense probe.

In one embodiment, the target sequence is HBV relaxed circular DNA (rcDNA). In one embodiment, the probe further comprises a detectable label at the 5′ end of the probe and a quencher on the 3′ end of the probe. In another embodiment, the detectable label is in close proximity with the quencher when the probe is not hybridized to the target sequence.

In one embodiment, the probe is denatured by heat and subsequently hybridized to the target sequence at an optimal annealing temperature. In yet another embodiment, the signal emitted when the probe hybridizes to the target sequence is a fluorescence signal. In one embodiment, no fluorescence is emitted when the probe does not hybridize to the target sequence.

In one embodiment, the detectable label is a fluorophore.

In one embodiment, the reference sample is a cell that has been cultured in media that does not comprise the therapeutic agent.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention and comparative examples will be further described in greater detail by reference to specific Examples, which should not be construed as in any way limiting the scope of the invention.

Materials and Methods

Plasmids

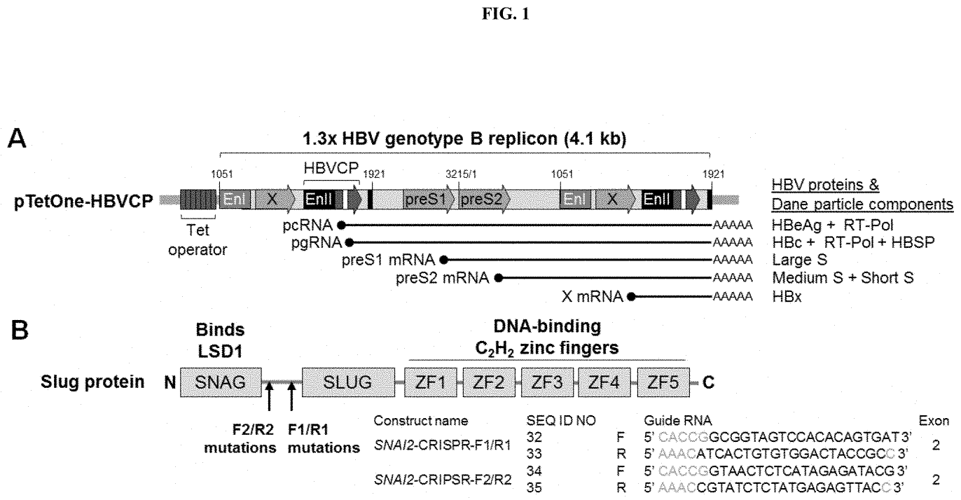

Luciferase reporter constructs for the HBVCP of genotypes A-H were generated by cloning into the PGL3 Basic (Promega) construct via Kpnland HindIII restriction sites. Differences in HBV replication efficiency between genotypes A-D were further ascertained using 1.3× replicons (SEQ ID NO: 1-4) inserted into pcDNA3.1+(ThermoFisher Scientific) via the MfeI and MluI restriction sites, with the CMV promoter specifically removed by excision using MluI and KpnI restriction sites. Thus HBV replication efficiency depends only on the activation of HBV promoters and enhancer elements. The inducible 1.3×HBV genotype B replicon pTetOne-HBVCP was inserted into pTetOne™ (Clontech) via NotI and MluI restriction sites, so that the Doxycycline-sensitive Tet operator is juxtaposed to the HBV promoter elements (Enhancer1-HBVCP) necessary for HBV replication (FIG. 1A).

CRISPR/Cas9 mediated targeting constructs for SNAI2 gene was generated in pX330. 2 constructs, SNAI2-CRISPR-F1/R1 and SNAI2-CRISPR-F2/R2 were generated (FIG. 1B), carrying sequences for guide RNA 1 (5′ GCGGTAGTCCACACAGTGAT 3′) (SEQ ID NO: 36) and guide RNA 2 (5′ GTAACTCTCATAGAGATACG 3′) (SEQ ID NO: 37) respectively targeting the 5′ end of exon 2 of human SNAI2 (NM_003068.4). Successful gene editing therefore generates a truncation mutant that renders the protein dysfunctional, as truncated Slug can no longer bind DNA without its C₂H₂ zinc fingers.

Cells and Culture Conditions

HuH7 cells were grown in DMEM (Gibco) supplemented with 10% Fetal bovine serum (FBS) (Gibco) in a humid incubator at 37° C. with 5% CO₂ supply prior to transfection. 5×10⁵ HuH7 cells were stably transfected in 6-well plates with 1.7 μg each of SNAI2-CRISPR-F1/R1, SNAI2-CRISPR-F2/R2 and pTetOne-HBVCP, along with 250 ng of linear hygromycin marker (Clontech) for subsequent clone selection in 500 μl of OPTI-MEM (Gibco) and 5.5 μl of Lipofectamine2000 (ThermoFisher Scientific). Culture media was changed and supplemented with minimal lethal dose of 200 μg/ml hygromycin in DMEM containing 10% Tet-free FBS (Clontech) 48 hours post-transfection. Cell death was monitored and culture media changed every 48 h. To minimize false positives, the surviving transfected cells were allowed to grow with increased hygromycin concentration at 250 μg/ml 7 days post-transfection, which was further increased to 350 μg/ml at 10 days post-transfection till no further cell death was observed. The cells were then re-seeded into 96-well plates by limiting dilution in 100p culture media supplemented with 20% Tet-free FBS and 30% conditioned medium harvested from HuH7 cells grown to 50% confluence in 10% Tet-free FBS-DMEM. A series of 7 dilutions were performed such that cells were diluted down from 64 to 0.5 cells/well. The cells were grown for another 4-6 weeks, and healthy clones microscopically examined to contain only 1 clone per well from wells containing 0.5 to 4 cells/well were selected for upscale and storage in liquid nitrogen.

HBV Assays

Cell clones were thawed and grown in 10% Tet-free FBS-DMEM. To screen for HBV-integrated clones, each recovered clone was seeded in 96-well plates in duplicates, induced with 100 ng/ml Doxycycline or DMSO control in 100 μl DMEM containing 10% Tet-Free FBS for 6 days, with a change in culture media every 2 days to maintain Doxycycline levels. To quickly evaluate if a clone contained integrated HBV and was able to generate HBV, quantitative real-time PCR for rcDNA in HBV virions was performed using 1 μl of culture media in 10 μl reactions with LightCycler® 480 SYBR Green I Master (Roche) on the LightCycler® 480 (Roche). The primers used to detect HBV genotype B rcDNA were rcFB: 5′ TTCTTTCCCGATCACCAGTTGGACCC 3′ (SEQ ID NO: 38) and rcRB: 5′ CCCACCTTGTTGGAGTCCGGC 3′ (SEQ ID NO: 39). The qPCR conditions are as follows: 1 cycle of 95° C. for 10 minutes to boil and release rcDNA from HBV particles, 40 rounds of 95° C. for 30 s, 60° C. for 20 s and 72° C. for 20 s. Fluorescence was acquired at the end of each round at 80° C. HBV-containing samples generate a single amplicon with Tₘ at 84° C., and only Doxycycline-induced clones with >10-fold increase in rcDNA when compared with DMSO controls were further evaluated. Amongst these, clones with rcDNA copies <10⁵/ml media in the presence of Doxycycline were also not evaluated. Selected HBV-producing clones were re-evaluated periodically over 6 months in the manner as described above in 24-well plates, and HBV-integration affirmed by positive immunofluorescence staining for HBc and HBs.

To determine relative strength of HBVCP activation between genotypes, luciferase reporter assays (Promega) were performed in 96-well clear-bottom black well plates. 2×10⁴ HuH7 cells were transfected with 0.2 μg HBVCP reporter constructs using 0.22 μl Lipofectamine2000 and 20 μl OPTI-MEM and the relative amounts of luciferase generated determining luminescence emitted at indicated time-points. Full-length replicons in pcDNA3.1+ lacking the CMV promoter (1 μg) were also transfected into 1×10⁵ HuH7 cells in 24-well plates using 1.1 μl Lipofectamine2000 and 100p of OPTI-MEM, and the resultant secretion of HBs and HBeAg into the culture media traced using ELISA with Monolisa™ HBsAg ULTRA (BioRad) and QuickTiter™ Hepatitis B “e” antigen (HBeAg) ELISA (Cell Biolabs) kits respectively.

Confirmation of SNAI2 Gene Knockout

gDNA from selected single cell clones were extracted using the Nucleospin® Tissue kit (Machery Nagel), and the SNAI2 gene fragment spanning intron 1 and exon 2 was amplified using the primer pair SNAI2-Intron-F2 (5′ TTACCAGTGTGTATGCCCTCCTAAATGG 3′) (SEQ ID NO: 40) and SNAI2-Exon2-R2 (5′ CCAGGCTCACATATTCCTTGTCACAG 3′) (SEQ ID NO: 41). The resultant PCR product was subjected to agarose gel electrophoresis and purified using the QIAquick® Gel extraction kit (Qiagen), and sent for Sanger sequencing using sequencing primers Seq-SNAI2-Intron1-F1 (5′ CTCCTAAATGGGTCTATCTTCTTCC 3′) and Seq-SNAI2-Exon2-R1 (5′ ATATTCCTTGTCACAGTATTTACAGCTG 3′). Clones with nonsense mutations that disrupted Slug coding sequence on both strands of DNA were deemed to have successful SNAI2 gene editing resulting in Slug knockout. As the protein and RNA expression of Slug is sub-detection in HuH7 cells, western blot, immunofluorescence staining and PCR could not be performed to ascertain knockout status.

Hybridization Assay

Molecular beacon DNA probes (IDT) Each hairpin probe bears 2 covalent modifications, a reporter at the 5′ end with 5′ TYE™563 fluorophore and a specific quencher at the 3′ end with 3′ IowaBlack® RQ. The hairpin structure of the probe keeps fluorescence quenched in the absence of target sequences. HBV-specific probe sequences used are as indicated in Table 1.

Probes were added to 5 μl of culture media from HBV-generating cells and 2 μl of PCR buffer from Expand High Fidelity PCR System (Roche) in 20 μl reactions, then incubated in the PCR machine for 15 minutes at 95° C. to release rcDNA from virions and linearize DNA and probes, then incubated at 60° C. for 15 minutes followed by probe annealing at 40° C. for 1 hour. 5 μl of the reaction was then added to 95 μl of phosphate buffered saline in clear-bottom black-well plates, and fluorescence read using a plate reader (Tecan) with default settings at 549 nm/565 nm excitation/emission maxima.

TABLE 1
HBV-specific sequences of probes and their specific targets used in hybridization
assay.

| | | SEQ | | Predicted HBV targets (No. of sites) | | |
|---|---|---|---|---|---|---|
| | | ID | | pgRNA/ | | rcDNA (+) |
| No. | Probe name | NO | Probe sequence (5′→3′) | pcRNA | X mRNA | strand |
| 1 | HBV_UniR1_En1 | 25 | CGCAGTATGGATCGGCAGAG | Yes (1) | No | Yes* (+/−1) |
| 2 | HBV_UniR2_ | 26 | GCACAGCTTGGAGGCTTGAA | Yes (2) | Yes (1) | Yes (1) |
| | BCPpolyA | | CA | | | |
| 3 | HBV_UniR3_pS2 | 27 | GAGAAGTCCACCACGAGTCTAG | Yes (1) | No | Yes* (+/−1) |
| 4 | HBV_UniR4_pS2 | 28 | GATGAGGCATAGCAGCAGGATG | Yes (1) | No | Yes* (+/−1) |
| 5 | HBV_UniR5_EnII | 29 | CAGAGGTGAAGCGAAGTGCAC | Yes (1) | Yes (1) | Yes (1) |
| 6 | HBV_UniRpgRNA1_ | 30 | TCCCACCTTATGAGTCCAAGG | Yes (1) | No | Yes (1) |
| | ABC | | | | | |
| 7 | HBV_UniRpgRNA2_ | 31 | CCTTCCAAAGAGTATGTAAAT | Yes (1) | No | Yes (1) |
| | AB | | AATGTC | | | |

*Due to the incomplete synthesis of rcDNA (+) strand in HBV virions, these probes may not be able to detect all copies of rcDNA in the culture media.

### Example 1: HBV Genotype B Replicates Most Effectively in HuH7 Cells

The severity of HBV-associated liver disease has long known to be associated with virus genotype and patient ethnicity. Of the 10 major genotypes (A-J), genotypes B and C most prevalent in Asia lead to more severe disease outcomes such as hepatocellular carcinoma (HCC) and are associated with higher HBV titers. Hence, high HBV replication efficiency may depend on HBV genotype, which is in turn is dependent on the cell line used. Since the HBV core promoter (HBVCP, nt 1600-1860 of genotype A) is the main regulatory element controlling pgRNA synthesis hence early phase of HBV replication post-entry, this was tested by comparing HBVCP transcription activity of 8 genotypes (A-H) (SEQ ID NO: 5-12) in luciferase reporter assays.

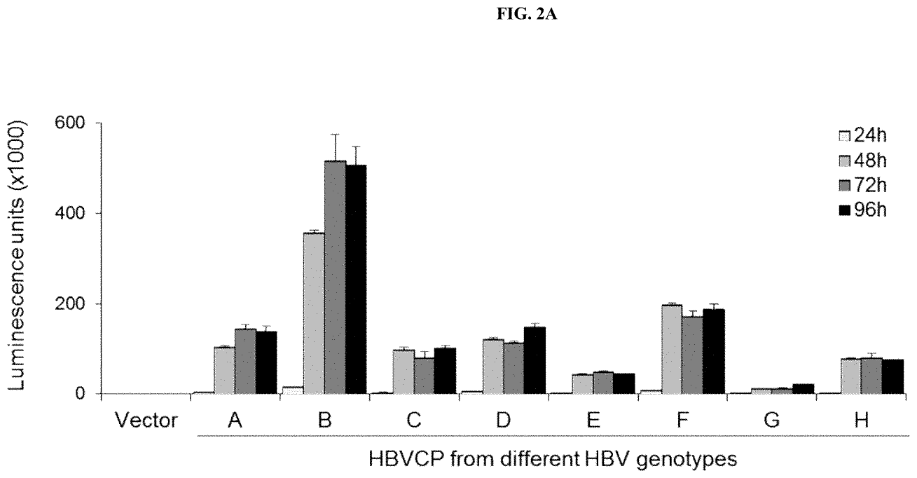

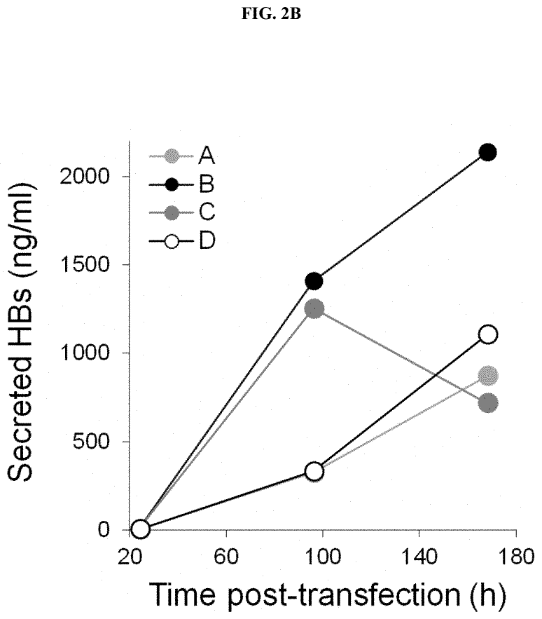

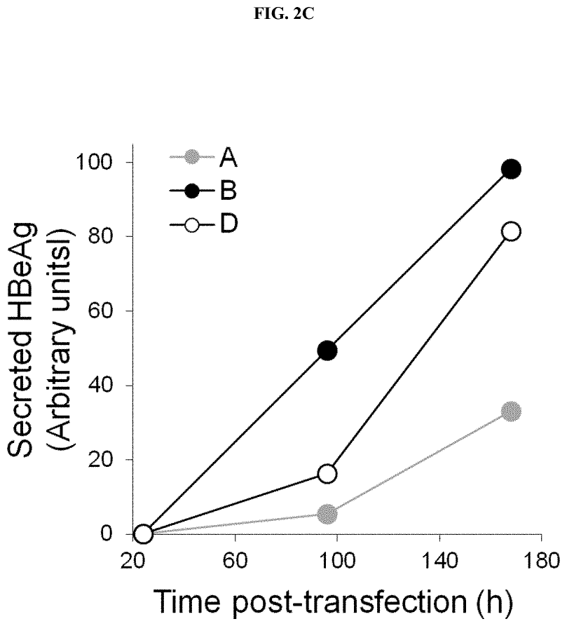

FIG. 2A shows that HBVCP transcription activity indeed differs greatly between genotypes, with highest activity in genotype B such that luminescence generated 96 hours post-transfection is 20× that of the weakest promoter in genotype G. This correlates well with known clinical outcomes of infection with genotype B virus, where such high burst of HBVCP transcription activity to generate more than twice the luminescence within 48 hours of what other genotypes can maximally achieve within 96 h post-transfection would increase the likelihood of patients presenting fulminant hepatitis and acute hepatitis (Shi, 2012). Genotype B is also known to be associated with HCC in younger patients <35 years of age. Interestingly, genotype C was a much weaker promoter despite multiple reports correlating it with chronic hepatitis and liver cancer, perhaps indicating that tolerance of lower levels of HBV which is not cleared by immune processes contributes to chronic infection and inflammation hence higher liver cancer rates. Genotypes A and D prevalent in Europe do not differ significantly from each other, and function at ˜30% capacity relative to genotype B. Genotype F associated with fulminant hepatitis B has slightly stronger HBVCP transcription activity than other genotypes, producing ˜40% luminescence relative to genotype B. Since HuH7 cells have high transfection efficiency >90%, the effect of differential transfection efficiency is not sufficient to account for the gross discrepancy in HBVCP transcription activity between genotypes. Thus, it was clear that HBV genotype B would be most efficient in generating HBV in HuH7 human liver cells.

To ascertain that genotype B is most suited for generating most HBV in HuH7 cells, full-length 1.3× replicons of genotypes A-D that synthesize viral particles by relying only on HBV promoters were generated. HuH7 cells were transfected with the replicons, and the relative amount of HBs secreted into the culture media was assessed by ELISA (FIG. 2B). Even though HBs production is independent of the HBVCP, genotype B secreted most HBs, suggesting that most HBV can be secreted when using this genotype in HuH7 cells. Genotype C surprisingly generated similar amounts of HBs at early time-points, but this was rapidly degraded or inhibited at late time-points of 170 h post-transfection, providing further confirmation that HBV genotype C is not suited for efficient replication in HuH7 cells. Genotypes A and D did not differ significantly in HBs secretion profile, both secreting ˜50% less HBs than genotype B. HBV genotype B is therefore thus far, the best genotype for efficient HBV replication.

Next, it was determined whether the relative amount of secreted HBeAg also differs by genotype as HBeAg is a clinically important proxy indicative of active HBV replication that is generated from full-length transcripts initiated at the HBVCP. FIG. 2C shows that amongst the 3 genotypes tolerated by HuH7, genotype B secretes most HBeAg. This is consistent with the findings in FIG. 2A that show that the HBVCP is most transcriptionally active in genotype B. HBV genotype D consistently secreted slightly more HBeAg than genotype A, correlating well with clinical data showing that HBV genotype D is associated with higher HBeAg⁺ rates in patients and hence more severe liver disease. Since HBV genotype B consistently generated more markers of active HBV replication—HBVCP transcription, HBs and HBeAg, HBV genotype B was selected for generating a Doxycycline-inducible construct for the stable transfection of HuH7 to generate large amounts of HBV.

### Example 2: Slug Knockout is Necessary to Overcome Cellular Limit for HBV Replication

From FIG. 2A, it is clear that regardless of HBV genotype, transcription activity reaches maxima between 48-72 h post-transfection as no further increase in luminescence is observed beyond that. This suggests that HuH7 cells have a mechanism that limits transcription at the HBVCP which acts after transcription activation is initiated. It has previously been shown that Slug and Sox7 are natural repressors of transcription at the HBVCP and are weakly expressed in HuH7. A very small amount of Slug protein may be found in HuH7, which can bind directly to the pgRNA initiator to block HBVCP transcription. Sox7 competes with the potent HBVCP activator HNF4α for binding in Enhancer II of the HBVCP, thereby preventing HNF4α-mediated transcription activation. Since neither RNA nor protein for Sox7 could be detected by western blot, Sox7 is unlikely the inhibitor that limited HBVCP transcription in HuH7. Instead, the presence of trace amounts of Slug in HuH7 was sufficient to prevent further HBVCP transcription in later time-points. Thus Slug knockout is necessary to sustain efficient HBV replication.

2 targeting constructs for SNAI2 were generated, where selected target sites were predicted to give highest chance of success and minimal off-targets. These target sites both act on the linker between the SNAG and SLUG domains (FIG. 1B), which when successfully disrupted would adversely affect the C₂H₂ zinc fingers downstream to prevent DNA binding and recognition on the cognate Slug motif in the HBVCP. Both targeting constructs were simultaneously co-transfected with the pTetOne-HBVCP construct carrying 1.3×HBV genotype B replicon juxtaposed to Tet operator element (FIG. 1A) that allows control of HBV synthesis by enhancing HBV replication only in the presence of Doxycycline. As none of the plasmids carry a selection marker, small amounts of linearized hygromycin marker were concurrently transfected into HuH7 cells seeded in 6-well plates. The transfected cells were then subjected to hygromycin selection 48 hours post-transfection, and the surviving cells re-seeded into 96-well plates to obtain single cell clones by limiting dilution. Healthy single cell clones obtained were then tested for ability to generate HBV by detecting for rcDNA in secreted HBV Dane particles in the culture media. This was found to be more conclusive than other high-throughput methods detecting for HBV proteins, as proteins may be synthesized from partially integrated replicons whereas rcDNA can only be reverse-transcribed from full-length 3.5 kb pre-genomic RNA (pgRNA) synthesized from transcription at the HBVCP. Presence of rcDNA in culture media therefore provides evidence for complete replicon integration, and demonstrates the ability of the cell clone to generate all components of HBV. The clones were re-seeded in 96-well plates in duplicates, with one replicate treated with 100 ng/ml Doxycycline inducer and the other treated with DMSO. Clones that generated the rcDNA-specific peak in melt-curve analysis from quantitative real-time PCR, and were inducible by Doxycycline to produce 10× more rcDNA than corresponding DMSO controls to reach >10⁵ rcDNA copies/ml were selected for further evaluation.

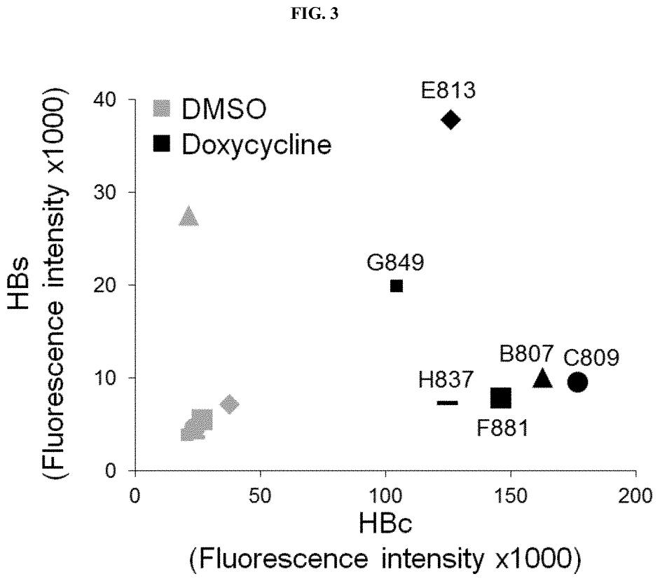

Only 6 clones fitted these criteria, and were further verified by positive immunofluorescence staining for the HBV nucleocapsid protein HBc and the HBV envelope protein HBs (FIG. 3). More importantly, the staining for HBc intensified with Doxycycline treatment, suggesting that full-length replicon was successfully integrated along with the Tet operator, which exerts significantly more effect on the HBVCP for HBc synthesis than the downstream preS2 promoter for HBs synthesis (FIG. 1A). Taken together, the Tet-On system works well on the HBVCP to control HBV replication efficiency in the presence of Doxycycline.

Clones C809 and F881 sustained HBV production for longer than 6 months in culture hence were further evaluated for SNAI2 gene status. Genomic DNA from these were extracted, the targeted region between Intron 1 and Exon2 amplified by PCR and sent for Sanger sequencing. The results revealed that clone C809 was unmodified at SNAI2 gene, whereas clone F881 was mutated in the amplified region at exactly where the CRISPR/Cas9 enzymes were designed to target. The nonsense mutations affected the Slug coding sequence, disrupting the C₂H₂ zinc fingers (FIG. 1B) which are necessary for recognizing the pgRNA initiator motif in the HBVCP. Thus, Clone F881 is the anticipated stable cell clone with integrated HBV genotype B genome in a Slug knockout (KO) HuH7 cell. Clone C809 may be used as a Slug wildtype (WT) reference to compare how clone F881 fares without Slug expression.

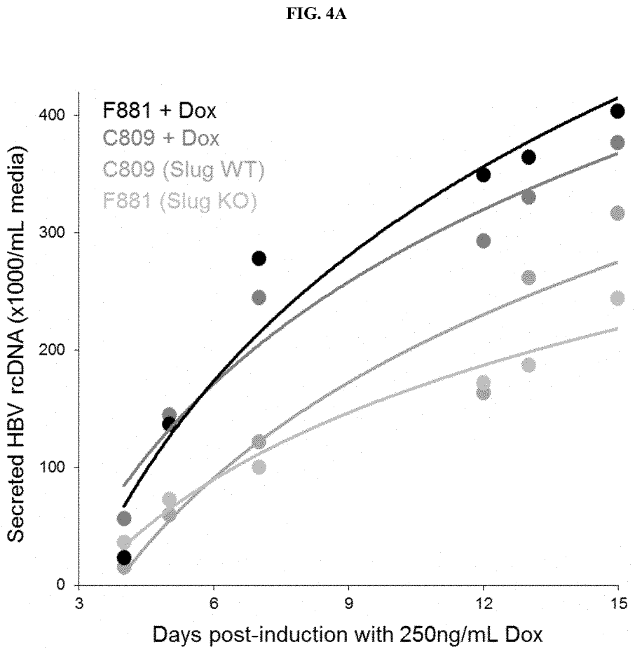

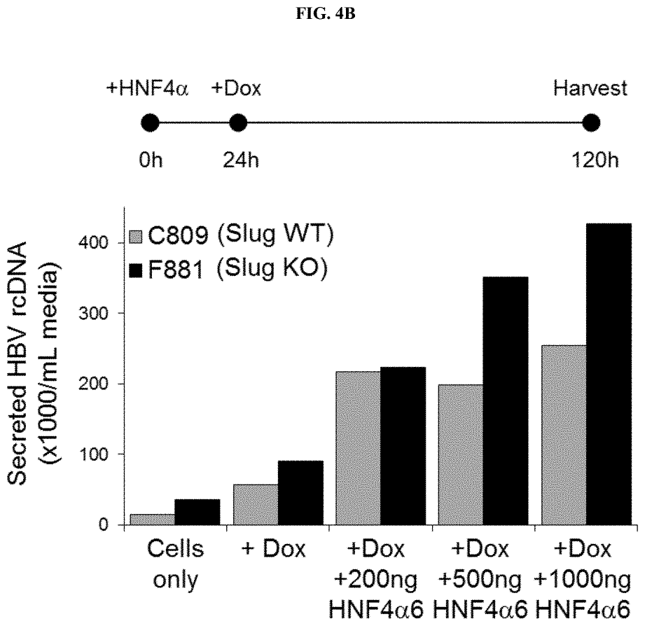

Both cell clones were studied in greater detail by tracking rcDNA copies in culture media when treated with or without optimal Doxycycline (Dox) dose (FIG. 4A). Both generated some HBV even when treated with DMSO only, with clone F881 being less productive in later time-points of >7 days induction. With continual media replacement every 48 hours to maintain optimal Dox dose of 250 ng/ml, the increase in HBV production in Clone F881 superseded that of Clone C809, suggesting that the loss of Slug can indeed enhance HBVCP transcription.

To further ascertain how the absence of Slug lifts HBVCP transcription restriction, it was tested whether overexpression of the most potent HNF4α isoform homodimer, HNF4α6 could further enhance HBVCP transcription to generate even more HBV. The clones were transfected with HNF4α overexpression constructs (SEQ ID NO: 13-24), and culture media changed for Dox induction 24 hours after transfection. As anticipated, in the presence of small amounts of Slug, clone C809 could be induced by Dox to generate more rcDNA, which was enhanced in the presence of HNF4α6. However, the amount of rcDNA generated reached a plateau that could not be overcome with increasing HNF4α6 overexpression (FIG. 4B). In stark contrast, the lack of functional Slug in clone F881 enabled the clone to generate more rcDNA without induction, which steadily increased with the addition of Dox and increased further in the presence of overexpressed HNF4α6. This increase in rcDNA remarkably continued to increase with higher amounts of overexpressed HNF4α6 without restriction to reach 400,000 copies/ml media within just 5 days of Dox induction compared to 15 days in the absence of HNF4α6 (FIG. 4A). Thus, the presence of tiny amounts of Slug in HuH7 is sufficient to greatly hinder HBV replication. Slug knockouts are required for a cell line to efficiently generate HBV.

### Example 3: HNF4α1-2 Isoform Heterodimer Maximizes Cellular Capacity to Generate HBV in Slug Knockouts

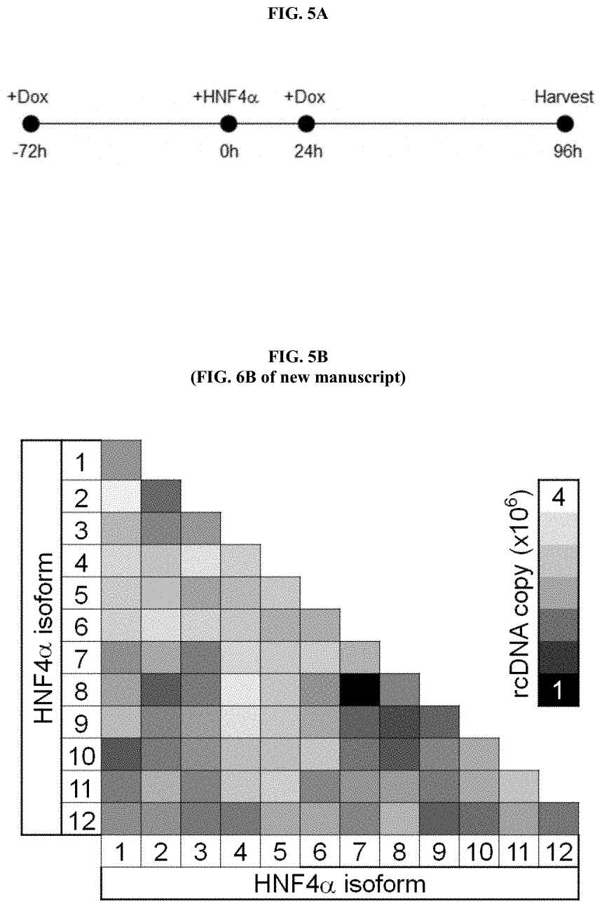

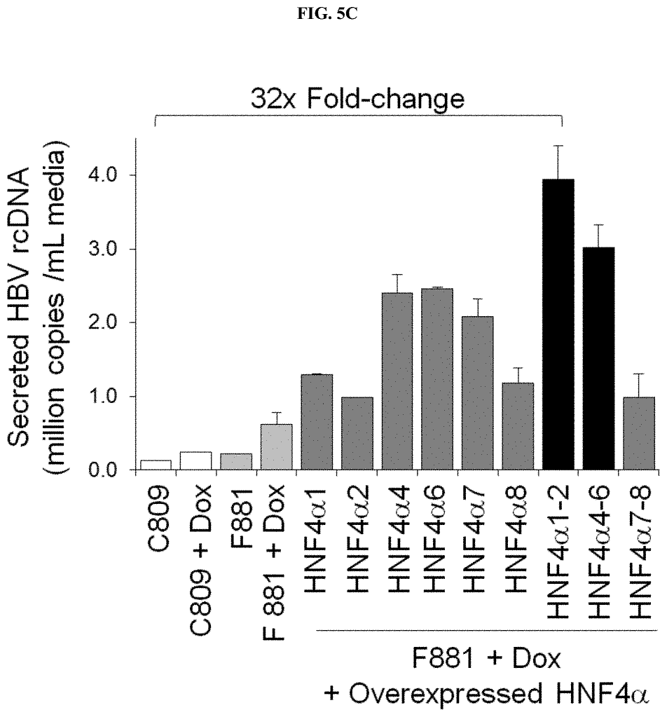

Transcription at the HBVCP is very sensitive to the combinations of HNF4α activator present in the cell, as HNF4α isoform homodimers and HNF4α isoform heterodimers exert grossly different effects at HNF4α target promoters. To determine which HNF4α isoform or isoform heterodimer will best support HBV replication in Clone F881, we overexpressed all potential pair-wise combinations of HNF4α isoforms after inducing HBV synthesis in the clone for 3 days, and continued the Dox treatment for another 3 days after 24 hours of transfection (FIG. 5A). Culture media was tested at 96 hours post-transfection for rcDNA expression, and FIG. 5B shows clearly the wide range in effect exerted by the HNF4α isoform combinations, such that HNF4α7-8 generated only 1 million copies of rcDNA/ml of culture media whereas this was significantly increased to 4 million copies when HNF4α1-2 is overexpressed instead. It is interesting to note that HNF4α1 and HNF4α2 are associated with normal adult liver cell functions, whereas HNF4α7 and HNF4α8 are associated with stem cells and cancers. Thus, by simply overexpressing HNF4α1-2 isoform heterodimer in Clone F881 with a Slug knockout background in the presence of optimal Dox dose, HBV generation was significantly enhanced to unprecedented levels exceeding millions of copies within 3 days, representing a 32× enhancement over Clone C809 which stably expresses HBV genotype B (FIG. 5C). This level of HBV production was found to be comparable to that from serum of patients with active HBV replication and liver disease, where HBV DNA copies varied between 0.5 to 4.5 million copies/ml. Thus, Clone F881 was found to be well-suited for studying mechanisms for HBV-associated diseases, and its high capacity to generate large amounts of HBV in a very short time-span makes it well-suited for a cell-based assay to screen for novel anti-HBV therapeutics in a high throughput manner.

### Example 4: Rapid Detection of rcDNA in Culture Media for High-Throughput Screening

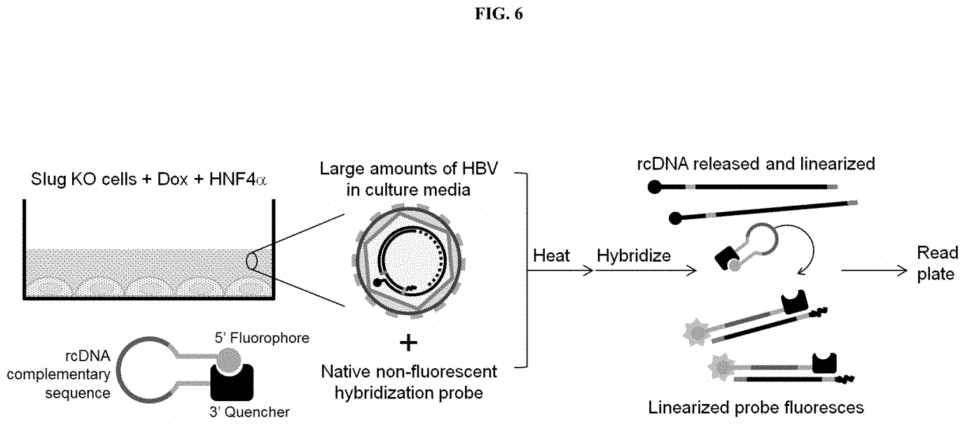

Since very high amounts of HBV load can be generated with Clone F881 under the right conditions of supplementation with Dox and overexpression of HNF4α1-2 isoform heterodimer, the rcDNA from culture media would be in sufficiently high quantities for detection using a simplified hybridization protocol without the need for signal amplification (FIG. 6). In such case, additional materials and enzymes for signal amplification and wash steps to remove excess probes and amplification materials would not be needed. Molecular beacon probes specific for rcDNA sequences can be designed to have a fluorophore attached at its 5′ end, and a quencher at the 3′ end. Fluorescence from the fluorophore would be specifically inhibited under normal circumstances by the quencher as the probe forms a hairpin to bring the 5′ fluorophore in close proximity to its quencher. When the probe is incubated with rcDNA in culture media and heated, the probe denatures and linearizes along with rcDNA, preventing the quencher from acting on the fluorophore. When optimal annealing temperature is reached, the fluorescent linearized probe can then bind to its target sequence in linearized single-stranded rcDNA by complementary base-pairing, allowing the bound probe to retain its linear conformation hence continue to fluoresce. Unbound probes would reform the hairpin structure once temperature drops further, allowing the quencher to act on the fluorophore once more. Thus, unbound probes need not be washed away, as they will not interfere with the specific fluorescence generated from rcDNA-bound probes. This highly simplified protocol would significantly reduce sample processing time as all that is required would be to add the probes and heat the plate in temperature cycling equipment such as the PCR machine, then detect the fluorescence emitted using conventional fluorescent plate readers.

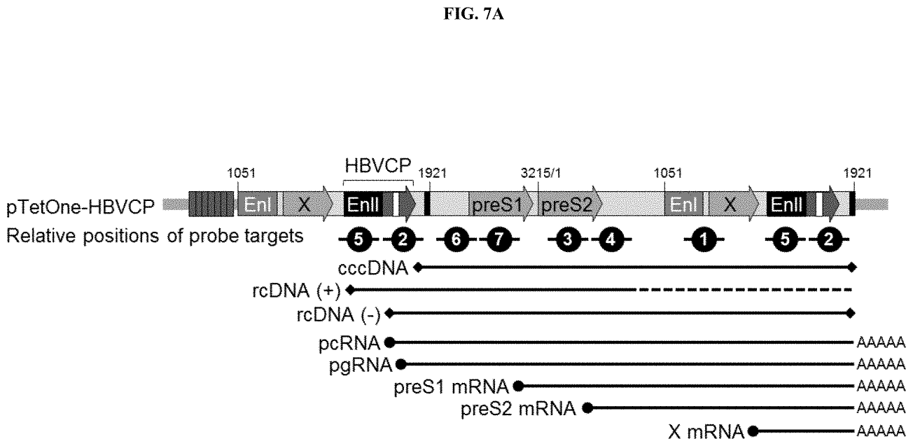

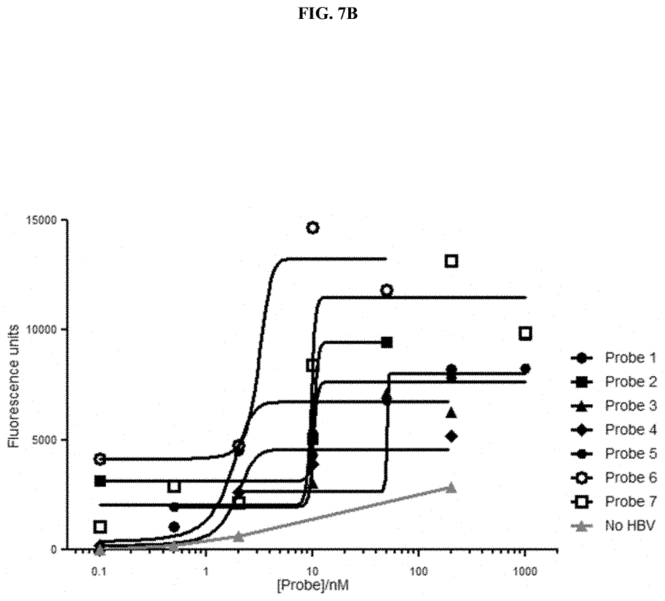

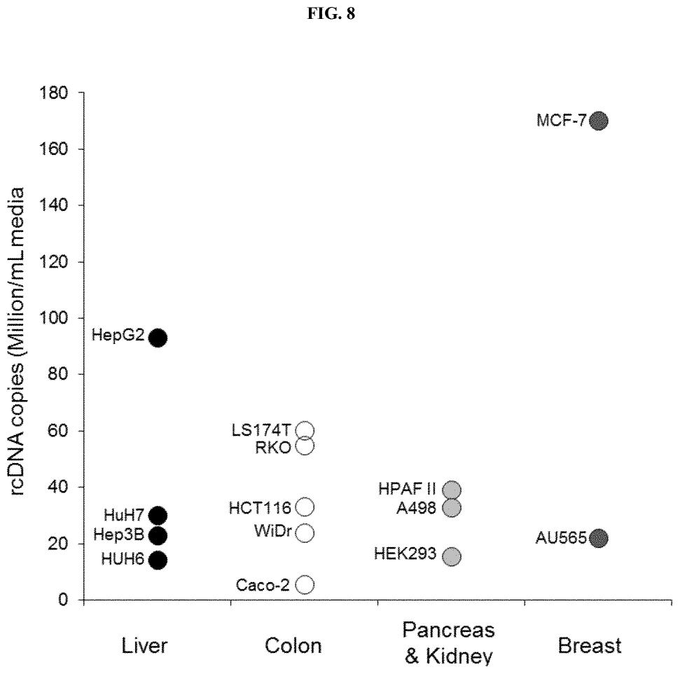

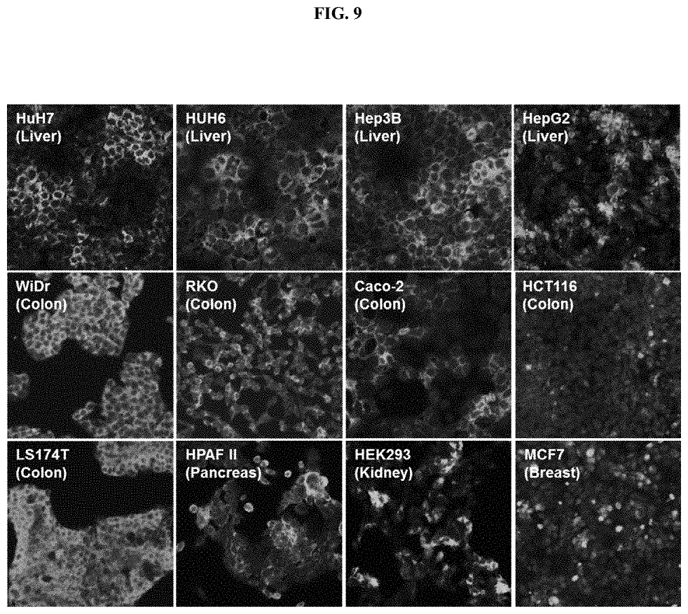

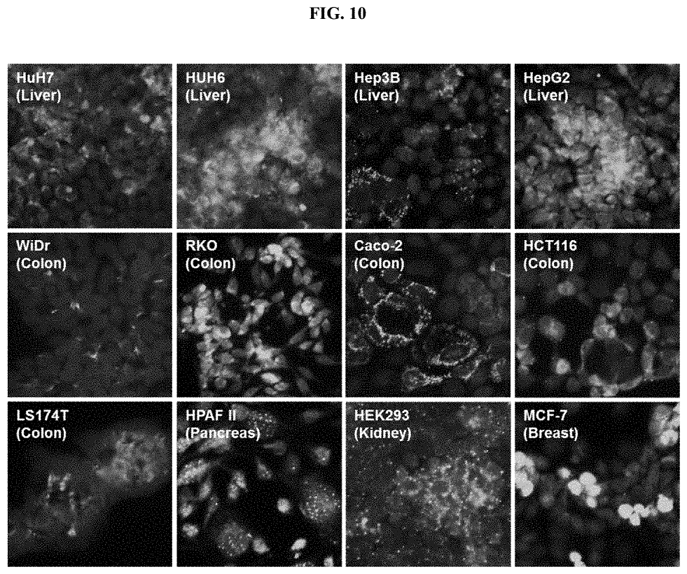

To test the feasibility of such a HBV detection method, 7 target probes were designed to target regions that are highly conserved within genotype B strains spread across the entire HBV genome (Table 1 and FIG. 7A). All the probes designed were anti-sense probes hence could only hybridize to the incomplete (+) strand of the partially double-stranded rcDNA. FIG. 7A shows the relative targets of the probes with reference to HBV transcripts, cccDNA and rcDNA. Since the (+) strand is incomplete, only probes 2, 5, 6 and 7 would bind to almost all rcDNA in the culture media, hence these probes reflect the actual attainable amount of rcDNA copies in the sample. Probes 3 and 4 lie within a region where the (+) strand synthesis ceases in infectious particles, hence even in excess, specific binding from these probes will yield less fluorescence than the probes 2, 5, 6 and 7. Probe 1 targets a region where synthesis for most (+) rcDNA strands would not reach, hence would generate the lowest fluorescence signal even if hybridization is successful. These probes have been designed to give varying maximum fluorescence signals, so that the success of the protocol would generate a range of fluorescence signals, and the failure of which would most likely give a homogenous signal regardless of probe sequence.

FIG. 7B shows the results of fluorescence generated with increasing probe concentration for culture media containing HBV. All the probes bound rcDNA specifically, as fluorescence was seen to increase with probe concentration and saturates between 3-40 nM, whereas in the culture media control containing no HBV, fluorescence remained low and unsaturated even at high probe concentration of 200 nM. Consistent with the (+) strand of rcDNA being incomplete, probes 1 and 4 had very low fluorescence even at saturation due to the lack of cognate binding sites. In contrast, probes 2, 6 and 7 target the region where the (+) strand of rcDNA is first synthesized hence generate high fluorescence from binding to almost all available rcDNA copies. Taken together, this simple and rapid method of detecting rcDNA in culture media can is feasible for detecting HBV for cell lines with high HBV titer such as Clone F881.

By using reference amounts of HBV rcDNA as standards, this protocol can be readily enhanced to allow for quantification of rcDNA copies by correlating fluorescence generated from known HBV rcDNA standards. This simple protocol allows for automation, which when combined with the cell clone F881 that generates large amounts of HBV with Dox and overexpression of HNF4α1-2, is well-suited for large-scale high throughput screening of vast chemical libraries for the much needed anti-HBV therapeutic.

### Example 5: HBV Replication in Hepatic and Non-Hepatic Cells

Liver and non-liver cells were infected with HBV genotype A. The quantity of HBV rcDNA generated 72 hours post-infection was determined by quantitative real-time PCR of rcDNA found in infectious particles from 1 μl of culture media. The sequences of primers used were rcFA: 5′ ttctttcccgatcatcagttggaccc 3′ (SEQ ID NO: 44) and rcRA: 5′ CCTACCTGGTTGGCTGCTGGC 3′ (SEQ ID NO: 45). Several non-liver cells generated equivalent or more rcDNA than the liver cell lines, indicating that they produce equivalent or higher HBV titres than the liver cells.

Liver and non-liver cells were infected with HBV genotype A. The cells stained positive for HBx 3 days post-infection and this is indicative of successful HBV entry into the cells. Staining positive for HBx is also indicative of cccDNA formation to allow transcription of HBV products.

Liver and non-liver cells were infected with HBV genotype A. The cells stained positive for HBc 7 days post-infection. This is indicative of continued active HBV replication and production.

TABLE 2
Summary of sequence listing.

| Name | Description | SEQ ID NO |
|---|---|---|
| HBV Genotype A | Nucleic acid sequence of 1.3x HBV replicon - | 1 |
| | Genotype A | |
| HBV Genotype B | Nucleic acid sequence of 1.3x HBV replicon - | 2 |
| | Genotype B | |
| HBV Genotype C | Nucleic acid sequence of 1.3x HBV replicon - | 3 |
| | Genotype C | |
| HBV Genotype D | Nucleic acid sequence of 1.3x HBV replicon - | 4 |
| | Genotype D | |
| HBVCP Genotype A | Nucleic acid sequence of HBVCP sequences - | 5 |
| | Genotype A | |
| HBVCP Genotype B | Nucleic acid sequence of HBVCP sequences - | 6 |
| | Genotype B | |
| HBVCP Genotype C | Nucleic acid sequence of HBVCP sequences - | 7 |
| | Genotype C | |
| HBVCP Genotype D | Nucleic acid sequence of HBVCP sequences - | 8 |
| | Genotype D | |
| HBVCP Genotype E | Nucleic acid sequence of HBVCP sequences - | 9 |
| | Genotype E | |
| HBVCP Genotype F | Nucleic acid sequence of HBVCP sequences - | 10 |
| | Genotype F | |
| HBVCP Genotype G | Nucleic acid sequence of HBVCP sequences - | 11 |
| | Genotype G | |
| HBVCP Genotype H | Nucleic acid sequence of HBVCP sequences - | 12 |
| | Genotype H | |
| HNF4α1 | Nucleic acid sequence of HNF4α1 | 13 |
| HNF4α2 | Nucleic acid sequence of HNF4α2 | 14 |
| HNF4α3 | Nucleic acid sequence of HNF4α3 | 15 |
| HNF4α4 | Nucleic acid sequence of HNF4α4 | 16 |
| HNF4α5 | Nucleic acid sequence of HNF4α5 | 17 |
| HNF4α6 | Nucleic acid sequence of HNF4α6 | 18 |
| HNF4α7 | Nucleic acid sequence of HNF4α7 | 19 |
| HNF4α8 | Nucleic acid sequence of HNF4α8 | 20 |
| HNF4α9 | Nucleic acid sequence of HNF4α9 | 21 |
| HNF4α10 | Nucleic acid sequence of HNF4α10 | 22 |
| HNF4α11 | Nucleic acid sequence of HNF4α11 | 23 |
| HNF4α12 | Nucleic acid sequence of HNF4α12 | 24 |
| HBV_UniR1_EnI | Nucleic acid sequence of probe | 25 |
| | HBV_UniR1_EnI | |
| HBV_UniR2_BCPpolyA | Nucleic acid sequence of probe | 26 |
| | HBV_UniR2_BCPpolyA | |
| HBV_UniR3_pS2 | Nucleic acid sequence of probe | 27 |
| | HBV_UniR3_pS2 | |
| HBV_UniR4_pS2 | Nucleic acid sequence of probe | 28 |
| | HBV_UniR4_pS2 | |
| HBV_UniR5_EnII | Nucleic acid sequence of probe | 29 |
| | HBV_UniR5_EnII | |
| HBV_UniRpgRNA1_ABC | Nucleic acid sequence of probe | 30 |
| | HBV_UniRpgRNA1_ABC | |
| HBV_UniRpgRNA2_AB | Nucleic acid sequence of probe | 31 |
| | HBV_UniRpgRNA2_AB | |
| SNAI2-CRISPR-F1/R1_F | Nucleic acid sequence of SNAI2-CRISPR- | 32 |
| | F1/R1 forward primer | |
| SNAI2-CRISPR-F1/R1_R | Nucleic acid sequence of SNAI2-CRISPR- | 33 |
| | F1/R1 reverse primer | |
| SNAI2-CRIPSR-F2/R2_F | Nucleic acid sequence of SNAI2-CRIPSR- | 34 |
| | F2/R2 forward primer | |
| SNAI2-CRIPSR-F2/R2_R | Nucleic acid sequence of SNAI2-CRIPSR- | 35 |
| | F2/R2 reverse primer | |
| Guide RNA 1 | Nucleic acid sequence of Guide RNA 1 | 36 |
| Guide RNA 2 | Nucleic acid sequence of Guide RNA 2 | 37 |
| rcFB | Nucleic acid sequence of forward primer rcFB | 38 |
| rcRB | Nucleic acid sequence of reverse primer rcRB | 39 |
| SNAI2-Intron-F2 | Nucleic acid sequence of SNAI2-Intron-F2 | 40 |
| | forward primer | |
| SNAI2-Exon2-R2 | Nucleic acid sequence of SNAI2-Exon2-R2 | 41 |
| | reverse primer | |
| Seq-SNAI2-Intron1-F1 | Nucleic acid sequence of forward primer Seq- | 42 |
| | SNAI2-Intron1-F1 | |
| Seq-SNAI2-Exon2-R1 | Nucleic acid sequence of reverse primer Seq- | 43 |
| | SNAI2-Exon2-R1 | |
| rcFA | Nucleic acid sequence of forward primer rcFA | 44 |
| rcRA | Nucleic acid sequence of reverse primer rcRA | 45 |

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A cell comprising:
a nucleotide sequence encoding a Hepatitis B Virus (HBV) operably linked to a promoter;
two nucleotide sequences each encoding an isoform of HNF4α; and,
a nucleotide sequence encoding a repressor of HBV transcription, wherein said nucleotide sequence is mutated to decrease or silence expression of the repressor.

2. The cell according to claim 1, wherein the HBV is of genotype B.

3. The cell according to claim 1, wherein the promoter operably linked to the nucleotide sequence encoding the HBV is an inducible promoter, optionally wherein the inducible promoter is a doxycycline inducible promoter.

4. (canceled)

5. The cell according to claim 1, wherein the nucleotide sequence encoding HBV operably linked to a promoter is stably integrated into the genome of the cell.

6. The cell according to claim 1, wherein the isoform of HNF4α is selected from the group consisting of HNF4α1, HNF4α2, HNF4α3, HNF4α4, HNF4α5, HNF4α6, HNF4α7, HNF4α8, HNF4α9, HNF4α10, HNF4α11, and HNF4α12.

7. The cell according to claim 1, wherein each of the two nucleotide sequences encodes the same isoform, or different isoforms of HNF4α, optionally wherein the two nucleotide sequences encode isoforms HNF4α1 and HNF4α2 (HNF4α1-2), HNF4α2 and HNF4α3 (HNF4α2-3), HNF4α3 and HNF4α4 (HNF4α3-4), HNF4α2 and HNF4α6 (HNF4α2-6), HNF4α3 and HNF4α8 (HNF4α0-8), HNF4α4 and HNF4α8 (HNF4α4-8), HNF4α4 and HNF4α9 (HNF4α4-9), HNF4α6 and HNF4α12 (HNF4α6-12).

8. (canceled)

9. The cell according to claim 1, wherein the two nucleotide sequences encode isoforms HNF4α1 and HNF4α2 (HNF4α1-2).

10. The cell according to claim 1, wherein the mutation of the nucleotide sequence encoding a repressor of HBV transcription is selected from the group consisting of insertion, deletion, substitution, or a combination thereof of one or more nucleotides.

11. The cell according to claim 1, wherein the repressor of HBV transcription is SLUG, optionally wherein the nucleotide sequence encoding SLUG is mutated at one or more positions encoding amino acid residues starting from position 56 of SLUG, optionally wherein the nucleotide sequence encoding a repressor of HBV transcription is mutated by a CRISPR-Cas9 system.

12.-13. (canceled)

14. The cell according to claim 1, wherein the cell is a hepatic cell, optionally wherein the cell is selected from the group consisting of HepG2, HuH7 and Hep3B.

15. (canceled)

16. The cell according to claim 14, wherein the cell is HuH7.

17. The cell according to claim 1, wherein the cell is a cell line.

18. (canceled)

19. A method to produce HBV in vitro comprising culturing the cell according to claim 1 in the presence of an inducer for regulating transcription of the promoter.

20. The method of claim 19, wherein the HBV is produced at an increased level compared to a baseline level.

21. A method of detecting the amount of HBV in a culture media in vitro comprising:
culturing the cell of claim 1 in a culture medium comprising an inducer for regulating transcription of the promoter;
contacting the cell with a probe capable of hybridizing to a target sequence on the HBV genome;
hybridizing the probe to the target sequence, wherein a signal is emitted when the probe hybridizes to the target sequence; and,
measuring the level of the emitted signal and comparing this to a signal from a reference sample to detect the amount of HBV in the culture media.

22. The method of claim 21, wherein the inducer is doxycycline and wherein the promoter is inducible by a Tet-on system.

23. The method of claim 21, wherein the probe comprises a nucleotide sequence that is complementary to the target sequence on the HBV genome.

24. The method of claim 21, wherein the probe further comprises a detectable label at the 5′ end of the probe and a quencher on the 3′ end of the probe.

25. (canceled)

26. The method of claim 21, further comprising identifying a HBV therapeutic agent
wherein a decrease in the emitted signal compared to the reference sample identifies the HBV therapeutic agent.

27. (canceled)

28. The method of claim 26, wherein the reference sample is a cell that has been cultured in media that does not comprise the therapeutic agent.
